(19) United States
(12) Patent Application Publication
(10) Pub. No.: US 2017/0014468 A1
(43) Pub. Date: Jan. 19, 2017

(54) METHODS OF USE FOR THERAPEUTICS TARGETING THE PATHOGEN PORPHYROMONAS GINGIVALIS

(71) Applicant: Cortexyme, Inc., South San Francisco, CA (US)

(72) Inventors: Stephen S. Dominy, Novato, CA (US); Casey Crawford Lynch, San Francisco, CA (US)

(21) Appl. No.: 14/699,898

(22) Filed: Apr. 29, 2015

Related U.S. Application Data
(60) Provisional application No. 62/060,437, filed on Oct. 6, 2014.
(60) Provisional application No. 61/986,022, filed on Apr. 29, 2014.
(60) Provisional application No. 62/020,304, filed on Jul. 2, 2014.

(51) Int. Cl. A61K 38/05; A61K 31/164; C07K 16/40

(52) U.S. Cl. CPC A61K 38/05; C07K 16/40; A61K 31/164; C07K 2317/76

(57) ABSTRACT

The invention relates to compositions, formulations, vaccines and antibodies for the prevention and/or treatment of aging and brain disorders, including Alzheimer's disease, diabetes, cardiovascular disease, retinal disorders and arthritis. The invention also provides methods of treatment of aging disorders, brain disorders, including Alzheimer's disease, diabetes, cardiovascular disease, retinal disorders, and arthritis by administering compositions, formulations, vaccines and antibodies described in the specification. The invention further provides methods for diagnosing or assessing risk of development of brain disorders in humans and animals. The invention further provides animal models for testing novel therapeutics for brain disorders.

The present application claims priority from U.S. Provisional Application Ser. No. 61/986,022, filed on Apr. 29, 2014, and U.S. Provisional Application Ser. No. 62/060,437, filed on Oct. 6, 2014.

### FIELD OF THE INVENTION

The invention relates to compositions, formulations, vaccines and antibodies for the prevention and/or treatment of aging and brain disorders, including Alzheimer's disease, diabetes, cardiovascular disease, retinal disorders and arthritis. The invention also provides methods of treatment of aging disorders, brain disorders, including Alzheimer's disease, diabetes, cardiovascular disease, arthritis, and retinal disorders by administering compositions, formulations, vaccines and antibodies described below. The invention further provides methods for diagnosing or assessing risk of development of brain disorders in humans and animals. The invention further provides animal models for testing novel therapeutics for brain and retinal disorders.

### BACKGROUND INFORMATION

Researchers have previously noted a correlation between periodontal disease and dementia, but have failed to establish a direct connection between brain disorders and a pathogenic moiety or methods for developing treatments or diagnostics based on a specific target.

Periodontal disease has been identified as a risk factor for several conditions including retinal disorders[1], stroke[2], cardiovascular disease[3], diabetes [4], retinal disorders [5] and rheumatoid arthritis[7]. Limited studies have investigated periodontal disease as a potential risk factor for dementia. A twin study conducted by Gatz et al.[8] found that those who had lost more than half their teeth at age 35 had a greater risk of developing Alzheimer's disease later in life. Stein et al.[9] also found a low number of teeth (9 or fewer) to be associated with increased risk of dementia in a subset of Nun Study participants. Using data from NHANES III, Noble et al.[10] found those participants with the highest levels of serum antibodies to periodontal bacteria to have significantly lower scores on delayed word recall and calculation tasks. Parks-Stein et al. found that elevated serum antibodies to P. gingivalis correlated to the later development of Alzheimer's but not to mild cognitive impairment[11].

Age has been shown to be strongly associated with the carriage of P. gingivalis in the oral cavity, with individuals 30-39 years-old having a carriage rate of 20%, and individuals 60-69 years of age having carriage rates of over 50%[12]. In individuals 60 years old and older, P. gingivalis has been shown to be significantly associated with cognitive impairment[10].

In addition to humans, aged dogs spontaneously develop many features of AD including cognitive decline and brain neuropathology[13]. Similar to humans, periodontal disease is one of the most common infectious diseases affecting adult dogs[14, 15]. Recently, using adult beagle dogs, researchers demonstrated the existence of Arg-gingipains in plaque samples taken from beagle dogs given a specific soft diet to increase plaque formation on tooth surfaces.[16]

### SUMMARY OF THE INVENTION

It is an object of the present invention to provide methods for treating or preventing a brain disorder.

It is another object of the present invention to provide compositions and/or methods for treating or preventing Alzheimer's disease.

It is another object of the present invention to provide compositions and/or methods for treating or preventing diabetes.

It is another object of the present invention to provide compositions and/or methods for treating or preventing cardiovascular diseases.

It is another object of the present invention to provide compositions and/or methods for treating or preventing rheumatoid arthritis.

It is another object of the present invention to provide compositions and/or methods for treating or preventing retinal disorders.

It is another object of the present invention to provide compositions and/or methods for identifying an individual having, possibly having, or at risk of developing, Alzheimer's disease or other brain disorder.

It is yet another object of the present invention to provide animal models for the discovery of pharmaceutical agents (therapeutics) targeting the brain.

In accordance with the above objects, the present invention is directed in part to a method of treating a brain disorder (e.g., a neurodegenerative disease (e g, Alzheimer's disease, Down's syndrome, epilepsy, autism, Parkinson's disease, essential tremor, dementias (e.g., fronto-temporal dementia, dementia with Lewy Body disease), progressive supranuclear palsy, amyotrophic lateral sclerosis, Huntington's disease, multiple sclerosis, mild cognitive impairment, age associated memory impairment, chronic traumatic encephalopathy, Lewy Body disease, multiple system atrophy), (e.g., a traumatic injury (e.g., traumatic brain injury, stroke)), (e.g., psychiatric disorder (e.g., schizophrenia, depression, etc.)), comprising administering to a subject in need thereof a therapeutically effective amount(s) of one or more of the following: (i) one or more compound(s) of Formula I, II, III, or IV, as defined below, or a pharmaceutically acceptable salt(s) of any of the foregoing; (ii) an antibacterial agent which is bacteriostatic or bacteriocidal with respect to P. gingivalis; (iii) one or more antibodies which bind to RgpA and/or RgpB and/or Kgp and/or other P. gingivalis proteins; (iv) an active vaccine comprising a P. gingivalis protein other than RgpA and/or RgpB and/or Kgp, or an immunogenic fragment thereof; (v) naturally and unnaturally occurring antimicrobial peptides; (vi) malabaricone C; (vii) polyphenolic compounds; (viii) 42 FA-70C1; (ix) a lysine derivative; (x) an arginine derivative; (xi) a compound selected from the group consisting of histatin 5, baculovirus p35, a single point mutant of cowpox viral cytokine-response modifier (CrmA (Asp>Lys)), phenylalanyl-ureido-citrullinyl-valyl-cycloarginal (FA-70C1), (acycloxy)methyl ketone (Cbz-Phe-Lys-CH₂OCO-2,4,6-Me₃Ph), peptidyl chloro-methyl ketones (e.g., chloromethyl of arginine, chloromethyl ketones of lysine, etc.); fluoro-methyl ketones, bromo-methyl ketones, ketopeptides, 1-(3-phenylpropionyl)piperidine-3(R,S)-carboxylic acid [4-amino-1(S)-(benzothiazole-2-carbonyl)butyl]amide (A71561), azapeptide fumaramide, aza-peptide Michael acceptors (e.g., Aza-Orn and Aza-Lys Michael acceptors, (PhCH₂CH₂CO-Leu-ALys-CHCHCOOEt, etc.), benzamidine compounds (e.g., pentamidine, benzamidine, bis-benzamidine derivatives with a pentamidine-related structure, 2,6-bis-(4-amidinobenzyl)-cyclohexanone, etc.), phenylalanyl-ureido-citrullinyl-valinyl-cycloarginal (C₂₇H₄₃N₉O₇) (FA-70C1), acyclomethylketone, activated factor X inhibitors (e.g., DX-9065a), cranberry nondialysable fraction, cranberry polyphenol fraction, pancreatic trypsin inhibitor, Cbz-Phe-Lys-CH₂O—CO-2,4,6-Me₃-Ph, E-64, chlorhexidine, zinc (e.g., zinc acetate); and mixtures thereof; and (xii) combinations of any of the foregoing. Methods of this embodiment of the present invention encompass administration of any one or all of the foregoing in combination with one or more additional therapeutic agents indicated for the treatment and/or prevention of brain disorders (e.g., Alzheimer's disease). The agent(s) preferably reduce(s) the circulating activity or level(s) of active RgpAand/or RgpB and/or Kgp and/or the activity or levels of active RgpB and/or Kgp in the brain and/or pathological effects of active RgpA and/or RgpB and/or Kgp in the subject, and therefore may be useful for the prevention and/or treatment of aging and brain disorders, including Alzheimer's disease, diabetes, cardiovascular disease, and arthritis in the subject.

In some of the embodiments, the pharmaceutically acceptable agent is preferably selective for RgpA and/or RgpB and/or Kgp such that it does not affect or substantially affect activity of human proteases other than RgpA and/or RgpB and/or Kgp when administered at a therapeutically effective dose for the treatment of brain disorders (e.g., AD), diabetes, cardiovascular disease, arthritis or retinal disorders by reducing the circulating activity or level(s) of active RgpB and/or Kgp and/or the activity or levels of active RgpA and/or RgpB and/or Kgp in the brain of a mammal, including humans and dogs. In certain embodiments, the pharmaceutically acceptable agent is preferably selective only for RgpA, In certain embodiments, the pharmaceutically acceptable agent is preferably selective only for RgpB, In certain embodiments, the pharmaceutically acceptable agent is preferably selective only for Kgp.

In some of the embodiments, the pharmaceutically acceptable agent is at least 30 times for selective for RgpA and/or RgpB and/or Kgp than for trypsin and cathepsin L (e.g., the Ki for Kgp is 0.9 nM and the Ki for trypsin is 30 nM or more and Ki for cathepsin L is 30 nM or more (e.g., 115 μM). Preferably, the pharmaceutically acceptable agent(s) of the invention does not adversely affect the coagulation cascade.

The present invention is also directed in part to a method of treating diabetes, cardiovascular disease, and arthritis comprising administering to a subject in need thereof a therapeutically effective amount of a pharmaceutically acceptable agent or compound which has a Ki of Arginine Gingipain A (RgpA), Arginine Gingipain B (RgpB) and/or Lysine Gingipain (Kgp) of less than 10 nanomolar (nM). For example, the pharmaceutically acceptable agent may have the Ki of from about 1 picomolar (pM) to about 10 nanomolar (nM), from about 10 pM to about 10 nM, from about 100 pM to about 10 nM, or from about 100 pM to about 9 nM. Preferably, the pharmaceutically acceptable agent is at least 30 times for selective for RgpA and/or RgpB and/or Kgp than for trypsin and cathepsin L (e.g., the Ki for Kgp is 0.9 nM and the Ki for trypsin is 30 nM or more and Ki for cathepsin L is 30 nM or more (e.g., 115 μM). Preferably, the pharmaceutically acceptable agent(s) of the invention does not adversely affect the coagulation cascade.

Administration of the pharmaceutically acceptable agent may reduce neurotoxic effects of RgpA and/or RgpB and/or Kgp, e.g, by reducing the circulating activity or level(s) of active RgpA and/or RgpB and/or Kgp and/or the activity or levels of RgpB and/or Kgp in the brain. Administration of the compound may also enhance the phagocytotic capacity of granulocytes to P. gingivalis thereby further reducing the circulating activity or level(s) of active RgpA and/or RgpB and/or Kgp and/or the activity or levels of RgpB and/or Kgp in the brain. The pharmaceutically acceptable agent is preferably selective for RgpA and/or RgpB and/or Kgp such that it does not affect or substantially affects activity of proteases other than RgpA and/or RgpB and/or Kgp when administered at the therapeutically effective amount, and, in the preferred embodiments, is neuroprotective.

The present invention is specifically directed in part to a method of treating Alzheimer's disease comprising administering to a subject in need thereof a therapeutically effective amount of one or more compound(s) of Formula I, II, III, IV, or pharmaceutically acceptable salt(s) thereof, as defined below. The pharmaceutically acceptable agent may be selective for Kgp (Ki of 10 nM or less) and show no inhibition or substantially no inhibition (e.g., no therapeutically significant inhibition) of RgpB and/or Kgp at concentrations up to 90 μM. The pharmaceutically effective agent may also be selective for RgpB (Ki 10 nM or less) and show no inhibition or substantially no inhibition (e.g., no therapeutically significant inhibition) of RgpA and/or Kgp at concentrations up to 90 μM. In yet other embodiments, the pharmaceutically effective agent may also be selective for RgpA (Ki 10 nM or less) and show no inhibition or substantially no inhibition (e.g., no therapeutically significant inhibition) of RgpB and/or Kgp at concentrations up to 90 μM.

In certain preferred embodiments, the pharmaceutically acceptable agent(s) the pharmaceutically acceptable agent(s) preferably have a Ki of Kgp of less than 10 nanomolar (nM), less than 8 nM, less than 6 nM, or less than 4 nM.

The present invention is also directed in part to a method of treating a brain disorder (e.g., a neurodegenerative disease), comprising administering to a subject in need thereof a therapeutically effective amount(s) of an antibiotic(s) to reduce the circulating activity or level(s) RgpB and/or Kgp and/or the activity or levels of RgpB and/or Kgp in the brain of a mammal (e.g., a human or an animal (e.g., a dog)). In certain embodiments, the antibiotic(s) reduces the circulating activity or level of RgpA, in addition to or instead of RgpB and/or Kgp. The antibiotic has inhibitory activity against gram negative bacteria. The antibiotic may, for example, be a quinolone (e.g., gemifloxacin, ciprofloxacin, oflaxacin, trovafloxacin, sitafloxacin, etc.), a β-lactam (e.g., a penicillin (e.g., amoxicillin, amoxacilin-clavulanate, piperacillin-tazobactam, penicillin G, etc.), a cephalosporin (e.g., ceftriaxone, etc.)), a macrolide (e.g., erythromycin, azithromycin, clarithromycin, etc.), a carbapenem (e.g., doripenem, imipenem, meropinem, ertapenem, etc.), a thiazolide (e.g, tizoxanidine, nitazoxanidine, RM 4807, RM 4809, etc.), a tetracycline (e.g., tetracycline, minocycline, doxycycline, eravacycline, etc.), clindamycin, metronidazole, satranidazole, an agent that inhibits/interferes with formation of a biofilm of anaerobic gram negative bacteria (e.g., oxantel, morantel or thiabendazole, etc), and combinations of two, three, four, five, six, or more of any of the foregoing agents. In certain embodiments, a combination of a penicillin (e.g., amoxicillin) and metronidazole or a combination of penicillin (e.g., amoxicillin), metronidazole and a tetracycline is used. Chlorhexidine (e.g., chlorhexidine digluconate), alone or in combination with Zn (e.g., zinc acetate), may also be used in combination with the administered antibiotics. The antibiotic may have an MIC₅₀ of P. gingivalis of less than 25 μg/ml. For example, the antibiotic may have an MIC₅₀ of P. gingivalis of less than 20 μg/ml, less than 15 μg/ml, less than 10 μg/ml, less than 8 μg/ml, less than 6 μg/ml, or less than 5 μg/ml. In certain embodiments, the antibiotic may have an MIC₅₀ of P. gingivalis of less than 1 μg/ml or less than 0.2 μg/ml.

In one embodiment, the invention provides a method of treating or preventing a brain disorder comprising administering to a subject in need thereof, one or more compound(s) of formula (I):

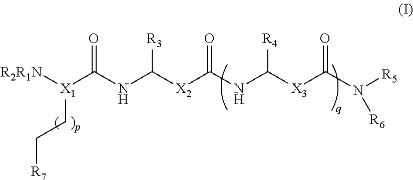

wherein

R¹, R², R⁵, and R⁶ is each independently a bond, hydrogen, an amino protecting group, hydroxyl, COOH, COH, carbonylaminoethylanilinyl, an alkyl (e.g., a lower alkyl (C₁-C₇)), a cycloalkyl, an alkenyl, an aryl (e.g., phenyl, benzyl, etc.), an alkylaryl, an arylalkyl (e.g., phenethyl), an alkoxyalkyl, an alkoxyaryl, an alkoxyalkylaryl, an alkoxyarylalkyl, an alkyloxycarbonyl, a carboxyalkyl, a carboxyaryl, a carboxyalkylaryl, a carboxyarylalkyl, a heterocycle radical (e.g., piperazinyl), an oxycarbonyl, benzyloxycarbonyl, amido, methylphenyamide, methylphenylamine, amine, carboxyl, alkyloxycarbonyl, or a side chain of an α-amino acid; the carbonylaminoethylanilinyl, the alkyl; the cycloalkyl, the alkenyl, the aryl, the alkylaryl, the arylalkyl, the alkoxyalkyl, the alkoxyaryl, the alkoxyalkylaryl, the alkoxyarylalkyl, the alkyloxycarbonyl, the carboxyalkyl, the carboxyaryl, the carboxyalkylaryl, the carboxyarylalkyl, the heterocycle radical (e.g., piperazinyl), the oxycarbonyl, the benzyloxycarbonyl, the amido, the methylphenyamide, the methylphenylamine, the amine, the carboxyl, the alkyloxycarbonyl, the side chain of the α-amino acid is each independently unsubstituted or substituted with one or more of amino, amide, halogen(s), hydroxyl, amitidine, a lower alkoxy (e.g., C₁-C₇), a lower carboxy (e.g., C₁-C₇), a lower alkyl (e.g., C₁-C₇), aryl (e.g., phenyl, benzyl, phenethyl), —NR⁸R⁹′ a loweralkoxycarbonylamino, (C₁-C₇), or a protecting group(s);

R³ and R⁴ is each independently a bond, hydrogen, hydroxyl, —COOH, —COH, —(CH₂)ₙ—NR⁸R⁹, an alkyl, an aminoalkyl, a cycloalkyl, an aryl, an alkylaryl, an arylalkyl, an alkoxyalkyl (a lower alkoxyalkyl (e.g., C₁-C₇)), an alkoxyaryl, an alkoxyalkylaryl, an alkoxyarylalkyl, a alkyloxycarbonyl, a carboxyalkyl, a carboxyaryl, a carboxyalkylaryl, a carboxyarylalkyl, a heterocycle radical (e.g., piperazinyl), oxycarbonyl, amido, carboxyl, guanidine, a side chain of an α-amino acid (e.g., lysine, arginine, etc.), isobutyl, carbamoylmethyl, 2-carboxyethyl, 4-aminobutyl, or benzyl; the hydroxyl, the —(CH₂)ₙ—NR⁸R⁹, the alkyl, the aminoalkyl, the cycloalkyl, the aryl, the alkylaryl, the arylalkyl, the alkoxyalkyl, the alkoxyaryl, the alkoxyalkylaryl, the alkoxyarylalkyl, the alkyloxycarbonyl, the carboxyalkyl, the carboxyaryl, the carboxyalkylaryl, the carboxyarylalkyl, the heterocycle radical (e.g., piperazinyl), the oxycarbonyl, the amido, the carboxyl, the guanidine, the side chain of the α-amino acid, the isobutyl, the carbamoylmethyl, the 2-carboxyethyl, the 4-aminobutyl, and benzyl is unsubstituted or substituted with one or more of amino, amide, halogen(s), hydroxyl, amitidine, a lower alkoxy (e.g., C₁-C₇), a lower carboxy (e.g., C₁-C₇), a lower alkyl (e.g., C₁-C₇), aryl (e.g., phenyl, benzyl, phenethyl), a loweralkoxycarbonylamino (C₁-C₇), or protective group(s);

R⁷ is a bond, hydrogen, hydroxyl, COOH, COR⁸, an alkyl (e.g., a lower alkyl (C₁-C₇)), a cycloalkyl, an aryl, an alkylaryl, an arylalkyl, an alkoxyalkyl (e.g., a lower alkoxyalkyl (e.g., C₁-C₇), e.g., methoxy, ethoxy, etc.), an alkoxyaryl, an alkoxyalkylaryl, an alkoxyarylalkyl, an alkyloxycarbonyl, a carboxyalkyl (e.g., a lower carboxyalkyl(C₁-C₇)), a carboxyaryl, a carboxyalkylaryl, a carboxyarylalkyl, a heterocycle radical (e.g., piperazinyl), oxycarbonyl, amino, methylamino, amido, dimethylamino, (2-aminoethyl)amino, 1,1-dimethylhydrazino, 1-methyl-1-phenylhydrazino, or benzyloxycarbonyl, a side chain of an α-amino acid, or carboxyl; the COR⁸, the alkyl, the cycloalkyl, the aryl, the alkylaryl, the arylalkyl, the alkoxyalkyl, the alkoxyaryl, the alkoxyalkylaryl, the alkoxyarylalkyl, alkyloxycarbonyl, the carboxyalkyl, the carboxyaryl, the carboxyalkylaryl, the carboxyarylalkyl, the heterocycle radical (e.g., piperazinyl), the oxycarbonyl, the amino, the methylamino, the amido, the dimethylamino, the (2-aminoethyl)amino, the 1,1-dimethylhydrazino, the 1-methyl-1-phenylhydrazino, the benzyloxycarbonyl, the carboxyl, the side chain of the α-amino acid is each independently unsubstituted or substituted with one or more of amino, amide, halogen(s), amitidine, hydroxyl, a lower alkoxy (e.g., C₁-C₇), a lower carboxy (e.g., C₁-C₇), a lower alkyl (C1-C7), —NR⁹R¹⁰, aryl (e.g., phenyl, benzyl, phenethyl), loweralkoxycarbonylamino, (C₁-C₇), or a protecting group(s);

R⁸, R⁹ and R¹⁰ is each independently a bond, hydrogen, hydroxyl, —COOH, —COH, —NH, —NH₂, —CNHNH₂, —CNH₂NNO₂, an alkyl (e.g., a lower alkyl (C₁-C₇)), a cycloalkyl, an aryl, an alkylaryl, an arylalkyl, an alkoxyalkyl (e.g., a lower alkoxyalkyl (C₁-C₇)), an alkoxyaryl, an alkoxyalkylaryl, an alkoxyarylalkyl, a carboxyalkyl, a carboxyaryl, a carboxyalkylaryl, a carboxyarylalkyl, a heterocycle radical (e.g., piperazinyl), oxycarbonyl, an alkyloxycarbonyl, an amino, hydrazine, or a side chain of an α-amino acid; the alkyl, the cycloalkyl, the aryl, the alkylaryl, the arylalkyl, the alkoxyalkyl, the alkoxyaryl, the alkoxyalkylaryl, the alkoxyarylalkyl, the carboxyalkyl, the carboxyaryl, the carboxyalkylaryl, the carboxyarylalkyl, the heterocycle radical (e.g., piperazinyl), the oxycarbonyl, the alkyloxycarbonyl, the hydrazine, the amino, the side chain of the α-amino acid independently unsubstituted or substituted with one or more of amino, amide, hydroxyl, halogen(s), amitidine, a lower alkoxy (e.g., C₁-C₇), a lower carboxy (e.g., C₁-C₇), a lower alkyl (C₁-C₇), aryl (e.g., phenyl, benzyl, phenethyl, etc.), loweralkoxycarbonylamino, (C₁-C₇) or a protective group(s);

X₁, X₂, X₃ is each independently a bond, —CH, —O—, —S, N, —CHOH—, —COO— or —CO—;

n is an integer from 1 to 6;

p is an integer from 0 to 4; and

q is an integer from 0 to 2,

or a pharmaceutically acceptable salt thereof. In certain embodiments, R¹ and/or R² may form a cyclic structure with R⁷. R⁵ and R⁶ may also form a cyclic structure in certain embodiments. In certain embodiments, one of R₃ and R₄ is the side chain of lysine the amino group of which may or may not be protected with a protecting group and the other of R3 and R4 is the side chain of argininine the guanidino group of which may or may not be protected with a nitro group.

In some of the embodiments, R¹ or R² is unsubstituted benzyloxycarbonyl or benzyloxycarbonyl substituted with one or more groups described above; R³ and R⁴ is each independently a bond, hydrogen, hydroxyl, carboxyl, an aminoalkyl, a side chain of an α-amino acid (e.g., lysine, arginine, etc.), the hydroxyl, the carboxyl, the aminoalkyl, the side chain of an α-amino acid may be unsubstituted or substituted with one or more groups discussed above; R⁵ and R⁶ is independently a bond, H, hydroxyl, carboxyl, a lower alkyl (e.g., C₁-C₇), alkylaryl (e.g., methylphenyl, ethylphenyl, etc.), the hydroxyl, the carboxyl, the lower alkyl, the alkylaryl may be unsubstituted or substituted with one or more group(s) described above; R⁷ is alkyl amine (e.g., methylamine, ethylamine, propylamine, butyl amine), 1-methyl-1-phenyl-hydrozinocarbonyl, alkylcarbonyl (e.g., propylcarbonyl), the alkyl amine, 1-methyl-1-phenyl-hydrozinocarbonyl, alkylcarbonyl may be unsubstituted or substituted with one or more groups described above; X₁ is CH, X₂ and X₃ are both CO, p is an integer from 1 to 4; and q is 1.

Administration of these compounds may result in at least a 20% reduction of circulating levels of active Arginine Gingipain A (RgpA), Arginine Gingipain B (RgpB) and/or Lysine Gingipain (Kgp) and/or a 20% reduction of circulating levels of active Arginine Gingipain A (RgpA) and/or Arginine Gingipain B (RgpB) and/or Lysine Gingipain (Kgp) in the brain. For example, after administration of these compounds, the circulating levels of active proteases and/or the levels of these active proteases in the brain may be reduced by from about 25% to about 95%, from about 35% to about 95%, from about 40% to about 85%, or from about 40% to about 80% as compared to the corresponding levels of these proteases 24 hours prior to the first administration of these compounds.

Methods of the present invention encompass administration of the compounds of Formula I in combination with one or more additional therapeutic agents indicated for the treatment and/or prevention of brain disorders (e.g., Alzheimer's disease).

The methods of treating or preventing a brain disorder in accordance with the present invention encompasses methods comprising administering to a subject in need thereof, one or more compounds of Formula II:

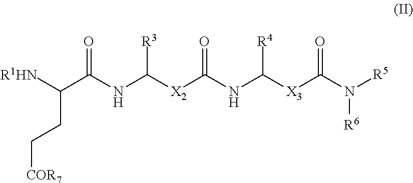

wherein X₂ and X₃ is each independently —CHOH— or —CO—; R¹ and R⁷ each is independently hydrogen and/or oxycarbonyl which may be unsubstituted or substituted with one or more group(s) described above; R³ is oxycarbonyl substituted with one or more of amino, amide, halogen(s), amitidine, hydroxyl, a lower alkoxy (e.g., C₁-C₇), a lower carboxy (e.g., C₁-C₇), —NR⁹R¹⁰, phenyl, benzyl, phenethyl, or a protecting group(s); R⁴ is hydroxyl, lower alkoxy (e.g. C₁-C₇), optionally substituted a six-membered heterocycle radical (e.g., piperazinyl); R⁵ is a R-group side chain of an α-amino acid optionally protected by a protective group, R⁶ is hydroxyl, lower alkoxy (e.g., C₁-C₇); m is 0 or 1; and n is an integer of 2 to 6; or a pharmaceutically acceptable salt thereof.

The methods of treating or preventing a brain disorder in accordance with the present invention also encompass methods comprising administering to a subject in need thereof, one or more compounds of Formula III

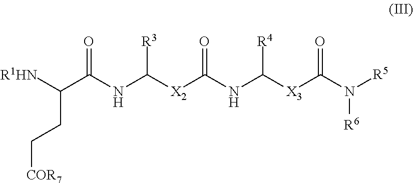

or a pharmaceutically acceptable salt thereof, wherein:
   X₂ and X₃ is each independently a bond, —CHOH— or —CO—;

R¹ and R⁷ is each independently hydrogen, an unsubstituted carboxyl group, a carboxyl group substituted with one or more group(s) described above, hydroxyl, an unsubstituted oxycarbonyl, or an oxycarbonyl substituted with one or more group(s) described above; R³ is an oxycarbonyl substituted with one or more group(s) described above, an unsubstituted alkyl, an alkyl substituted with one or more groups described above, an unsubstituted oxycarbonyl, an oxycarbonyl substituted with one or more groups described above, an unsubstituted aminoalkyl, an aminoalkyl substituted with one or more group(s) described above, or hydroxyl;

R⁴ is an unsubstituted alkyl, an alkyl substituted with one or more group(s) described above, an aminoalkyl, an aminoalkyl substituted with one or more groups described above, hydroxyl, a lower alkoxy (C₁-C₇), a six-membered unsubstituted heterocycle radical (e.g., a piperazinyl), a six-membered heterocycle radical (e.g., a piperazinyl) substituted with one or more group(s) described above;

R⁵ is an unsubstituted alkyl, an alkyl substituted with one or more group(s) described above, hydrogen or a R-group side chain of an α-amino acid optionally protected by a protective group;

R⁶ is an unsubstituted alkyl, and alkyl substituted with one or more group(s) described above, hydroxyl, or a lower alkoxy.

The methods of treating or preventing a brain disorder in accordance with the present invention also encompass methods comprising administering to a subject in need thereof, one or more compounds of Formula IV:

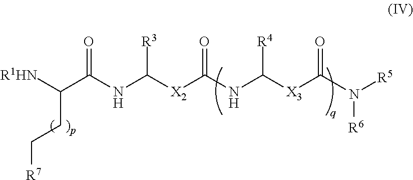

or a pharmaceutically acceptable salt thereof, wherein:
   R⁷ is an unsubstituted amino, an amino substituted with one or more substituents described above, —COR² or an amido optionally substituted with one or more groups described above;
   p is an integer from 1 to 4; and
   q is an integer from 0 to 2.

The present invention encompasses the use of compounds described in U.S. Pat. Nos. 7,067,476 and 6,951,843, herein incorporated by reference, for treating or preventing brain disorders (e.g., Alzheimer's Disease), diabetes, retinal disorders, arthritis and/or cardiovascular diseases comprising administering these compounds to a subject in need thereof. The invention also provides methods for improving systemic and brain delivery of compounds for treatment of brain disorders by administering one or more compound(s) of Formulas I, II, III, or IV, or mixtures thereof, to a subject in need thereof.

The present invention is also directed in part to a method of treating a brain disorder (e.g., a neurodegenerative disease (e.g., Alzheimer's disease, Down's syndrome, epilepsy, Parkinson's disease, essential tremor, dementias (e.g., fronto-temporal dementia, dementia with Lewy Body disease), progressive supranuclear palsy, amyotrophic lateral sclerosis, Huntington's disease, multiple sclerosis, mild cognitive impairment, age associated memory impairment, chronic traumatic encephalopathy, Lewy Body disease, multiple system atrophy), (e.g., a traumatic injury (e.g., traumatic brain injury, stroke)), (e.g., psychiatric disorder (e.g., schizophrenia, autism, depression, etc.)), diabetes, cardiovascular disease, retinal disorders and arthritis comprising administering to a subject in need thereof a therapeutically effective amount of a lysine derivative, an arginine derivative, histatin 5, baculovirus p35, a single point mutant of cowpox viral cytokine-response modifier (CrmA (Asp>Lys)), phenylalanyl-ureido-citrullinyl-valyl-cycloarginal (FA-70C1), (acycloxy)methyl ketone (Cbz-Phe-Lys-CH₂OCO-2,4,6-Me₃Ph), peptidyl chloro-methyl ketone(s) (e.g., chloromethyl of arginine, chloromethyl ketones of lysine, etc.); fluoro-methyl ketone(s), bromo-methyl ketone(s), ketopeptide(s), 1-(3-phenylpropionyl)piperidine-3(R,S)-carboxylic acid [4-amino-1(S)-(benzothiazole-2-carbonyl)butyl]amide (A71561), azapeptide fumaramide, aza-peptide Michael acceptor(s) (e.g., Aza-Orn and Aza-Lys Michael acceptors, (PhCH₂CH₂CO-Leu-ALys-CHCHCOOEt, etc.), benzamidine compound(s) (e.g., pentamidine, benzamidine, bis-benzamidine derivative(s) with a pentamidine-related structure, 2,6-bis-(4-amidinobenzyl)-cyclohexanone, etc.), phenylalanyl-ureido-citrullinyl-valinyl-cycloarginal (C₂₇H₄₃N₉O₇) (FA-70C1), acyclomethylketone, activated factor X inhibitors (e.g., DX-9065a), cranberry nondialysable fraction, cranberry polyphenol fraction, pancreatic trypsin inhibitor, Cbz-Phe-Lys-CH₂O—CO-2,4,6-Me₃-Ph, E-64, chlorhexidine, zinc (e.g., zinc acetate), or a combination of two, three or more of any of foregoing. In some of these embodiments, Zn may enhance potency and selectivity of the compounds (e.g., chlorhexidine, benzamidine, etc.) used in the methods of the invention.

Benzamidine compounds include, e.g., the following compounds and derivatives thereof:

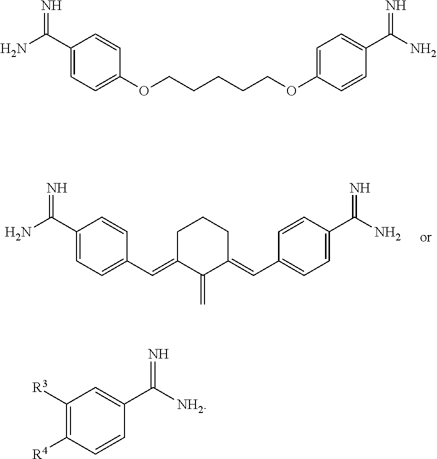

The present invention is also directed in part to a method of treating progressive supranuclear palsy, amyotrophic lateral sclerosis, Huntington's disease, mild cognitive impairment, age associated memory impairment, chronic traumatic encephalopathy, a psychiatric disorder (e.g., schizophrenia, autism, depression, etc.)), diabetes, cardiovascular disease, retinal disorders, and arthritis comprising administering to a subject in need thereof a therapeutically effective amount of an arginine derivative.

In yet another embodiment, the invention provides a method of treating diabetes comprising administering to a subject in need thereof, compounds in the same chemical series. Further, the invention provides a method of treating cardiovascular disease comprising administering to a subject in need thereof, one or more compound(s) in the same chemical series as Formulas I, II, III or IV. In yet another embodiment, the invention provides a method of treating retinal disorders comprising administering to a subject in need thereof, compounds in the same chemical series.

In certain embodiments, the pharmaceutically acceptable agent used in the methods of present invention is Kyt-1, Kyt-36, and/or Kyt-41.

One skilled in the art will appreciate that other peptides not specifically described in the chemical series as Formulas I, II, III or IV may also have inhibitory activity against P. gingivalis by themselves or synergistically in combination with another ingredient. For example, K-casein(109-107) is known to inhibit proteolytic activity associated with P. gingivalis whole cells, purified RgpA-gp proteinase-adhesion complexes, and purified RgpB. It has exhibited synergism with ZN(ii) against both Arg- and Lys-specific proteinases. Antimicrob Agents Chemother. 2011 March; 55(3):155-61. Administration of such inhibiting peptides, with or without synergistic ingredients such as (ZN_{(II)}) for the treatment of a brain disorder (e.g., a neurodegenerative disease (e g, Alzheimer's disease, Down's syndrome, epilepsy, Parkinson's disease, essential tremor, dementias (e.g., fronto-temporal dementia, dementia with Lewy Body disease), progressive supranuclear palsy, amyotrophic lateral sclerosis, Huntington's disease, multiple sclerosis, mild cognitive impairment, age associated memory impairment, chronic traumatic encephalopathy, Lewy Body disease, multiple system atrophy), (e.g., a traumatic injury (e.g., traumatic brain injury, stroke)), (e.g., psychiatric disorder (e.g., schizophrenia, autism, depression, etc.)), diabetes, cardiovascular disease, and arthritis is encompassed by the present invention.

The invention also provides a method of preventing or treating brain disorders by vaccination with proteins expressed by P. gingivalis other than Kgp and/or RgpA and/or RgpB, and immunogenic fragments thereof.

The invention also provides a method of preventing or treating brain disorders by administration of antibodies specific for gingipain epitopes or other proteins expressed by P. gingivalis.

The invention encompasses treatment with antibodies that bind to gingipains including, e.g., 18E6, 7B9, 61Bg 1.3, 1B5, 7B4, 15C8, humanized versions thereof, and other structurally or functionally similar antibodies. The RgpB-specific monoclonal antibody, 18E6, which binds to the first half of the immunoglobulin domain of RgpB[17]. The Kgp-specific monoclonal antibody, 7B9, recognizes an epitope within the Kgp catalytic domain[18]. The RgpA antibody 61Bg 1.3 has been shown in humans to reduce reinfection with P. gingivalis with respect to periodontal disease[19].

The invention also provides for the use of antibiotics and other bacteriocidal or bacteriostatic compounds with efficacy against P. gingivalis in the treatment of brain disorders.

The invention encompasses the use of antibiotics effective against P. gingivalis, alone and in combination with gingipain inhibitors and/or other treatments for Alzheimer's and other disorders. The compositions and methods of treatment may apply to treatment and prevention of brain disorders in mammals, including humans and animals (e.g., canines).

In an another aspect, the invention is directed in part to a method of treating diabetes comprising administering to a subject in need thereof a therapeutically effective amount(s) of (i) a pharmaceutically acceptable agent which inhibits Arginine Gingipain A (RgpA) and/or Arginine Gingipain B (RgpB) and/or Lysine Gingipain (Kgp) production, translocation of RgpB and/or Kgp into systemic circulation, and/or pathological effects of RgpA and/or RgpB and/or Kgp in a mammal; (ii) an antibacterial agent which is bacteriostatic or bacteriocidal with respect to P. gingivalis; (iii) one or more antibodies which bind to RgpA and/or RgpB and/or Kgp; (iv) a vaccine (passive or active) having specificity with respect to RgpA and/or RgpB and/or Kgp or other P. gingivalis proteins; and (v) combinations of any of the foregoing. Methods of this embodiment of the present invention encompass administration of any one or all of the foregoing in combination with one or more additional therapeutic agents indicated for the treatment and/or prevention of diabetes. The agent(s) preferably reduces the circulating activity or level(s) of active RgpA and/or RgpB and/or Kgp and/or the activity or levels of active RgpB and/or Kgp in the pancreas. In some of these embodiments, a therapeutically effective amount(s) of one or more compounds of formula I, II, III, and IV is administered alone or in combination with additional therapeutic agents indicated for the treatment and/or prevention of diabetes.

In yet another aspect, the invention is directed in part to a method of treating cardiovascular disease comprising administering to a subject in need thereof a therapeutically effective amount(s) of (i) a pharmaceutically acceptable agent which inhibits Arginine Gingipain A (RgpA) and/or Arginine Gingipain B (RgpB) and/or Lysine Gingipain (Kgp) activity or production, translocation of RgpB and/or Kgp into systemic circulation, and/or pathological effects of RgpA and/or RgpB and/or Kgp in a mammal; (ii) an antibacterial agent which is bacteriostatic or bacteriocidal with respect to P. gingivalis; (iii) one or more antibodies which bind to RgpA and/or RgpB and/or Kgp; (iv) a vaccine (passive or active) having specificity with respect to RgpA and/or RgpB and/or Kgp or other P. gingivalis proteins; and (v) combinations of any of the foregoing. Methods of this embodiment of the present invention encompass administration of any one or all of the foregoing in combination with one or more additional therapeutic agents indicated for the treatment and/or prevention of cardiovascular disease. The agent(s) preferably reduces the circulating activity or level(s) of active RgpA and/or RgpB and/or Kgp. In some of these embodiments, a therapeutically effective amount(s) of one or more compounds of formula I, II, III, and IV is administered alone or in combination with additional therapeutic agents indicated for the treatment and/or prevention of cardiovascular disease.

In yet another aspect, the invention is directed in part to a method of treating rheumatoid arthritis comprising administering to a subject in need thereof a therapeutically effective amount(s) of (i) a pharmaceutically acceptable agent which inhibits Arginine Gingipain A (RgpA) and/or Arginine Gingipain B (RgpB) and/or Lysine Gingipain (Kgp) activity or production, translocation of RgpB and/or Kgp into systemic circulation, and/or pathological effects of RgpA and/or RgpB and/or Kgp in a mammal; (ii) an antibacterial agent which is bacteriostatic or bacteriocidal with respect to P. gingivalis; (iii) one or more antibodies which bind to RgpA and/or RgpB and/or Kgp; (iv) a vaccine (passive or active) having specificity with respect to RgpA and/or RgpB and/or Kgp or other P. gingivalis proteins; and (v) combinations of any of the foregoing. Methods of this embodiment of the present invention encompass administration of any one or all of the foregoing in combination with one or more additional therapeutic agents indicated for the treatment and/or prevention of arthritis. The agent(s) preferably reduces the circulating activity or level(s) of active RgpA and/or RgpB and/or Kgp. In some of these embodiments, a therapeutically effective amount(s) of one or more compounds of formula I, II, III, and IV is administered alone or in combination with additional therapeutic agents indicated for the treatment and/or prevention of rheumatoid arthritis.

The invention is also directed to the compounds of formulas I, II, III, and IV for use in treatment of Alzheimer's disease, Down's syndrome, epilepsy, autism, Parkinson's disease, essential tremor, fronto-temporal dementia, progressive supranuclear palsy, amyotrophic lateral sclerosis, Huntington's disease, multiple sclerosis, mild cognitive impairment, age associated memory impairment, chronic traumatic encephalopathy, stroke, Lewy Body disease, multiple system atrophy, schizophrenia, depression, diabetes, cardiovascular disease, and/or rheumatoid arthritis.

The invention is further directed to a lysine derivative, an arginine derivative, histatin 5, baculovirus p35, a single point mutant of cowpox viral cytokine-response modifier (CrmA (Asp>Lys)), phenylalanyl-ureido-citrullinyl-valyl-cycloarginal (FA-70C1), (acycloxy)methyl ketone (Cbz-Phe-Lys-CH₂OCO-2,4,6-Me₃Ph), peptidyl chloro-methyl ketones (e.g., chloromethyl of arginine, chloromethyl ketones of lysine, etc.); fluoro-methyl ketones, bromo-methyl ketones, ketopeptides, 1-(3-phenylpropionyl)piperidine-3(R,S)-carboxylic acid [4-amino-1(S)-(benzothiazole-2-carbonyl)butyl]amide (A71561), azapeptide fumaramide, aza-peptide Michael acceptors (e.g., Aza-Orn and Aza-Lys Michael acceptors, (PhCH₂CH₂CO-Leu-ALys-CHCHCOOEt, etc.), benzamidine compounds (e.g., pentamidine, benzamidine, bis-benzamidine derivatives with a pentamidine-related structure, 2,6-bis-(4-amidinobenzyl)-cyclohexanone, etc.), phenylalanyl-ureido-citrullinyl-valinyl-cycloarginal (C₂₇H₄₃N₉O₇) (FA-70C1), acyclomethylketone, activated factor X inhibitors (e.g., DX-9065a), cranberry nondialysable fraction, cranberry polyphenol fraction, pancreatic trypsin inhibitor, Cbz-Phe-Lys-CH₂O—CO-2,4,6-Me₃-Ph, E-64, chlorhexidine, zinc (e.g., zinc acetate), or a combination of two, three or more of any of foregoing for in treatment of Alzheimer's disease, Down's syndrome, epilepsy, autism, Parkinson's disease, essential tremor, fronto-temporal dementia, progressive supranuclear palsy, amyotrophic lateral sclerosis, Huntington's disease, multiple sclerosis, mild cognitive impairment, age associated memory impairment, chronic traumatic encephalopathy, stroke, Lewy Body disease, multiple system atrophy, schizophrenia, depression, diabetes, cardiovascular disease, and/or rheumatoid arthritis.

The invention is further directed to an arginine derivative in treatment of autism, Parkinson's disease, essential tremor, progressive supranuclear palsy, amyotrophic lateral sclerosis, Huntington's disease, chronic traumatic encephalopathy, stroke, multiple system atrophy, schizophrenia, depression, diabetes, cardiovascular disease, and/or rheumatoid arthritis.

In an additional aspect, the present invention establishes the presence of gingipains in the brain of cognitively impaired dogs.

In a further aspect, the invention is directed in part to rodent and canine models for the discovery of therapeutics targeting the brain. In some of the embodiments, the invention is directed to mouse, rat and dog model of brain disorder are created by natural or artificial infection with P. gingivalis.

### DEFINITIONS

As used in the present specification the term “active protease” means a protein that can hydrolyze peptide bonds in a relevant substrate. For example, the term “active RgpB” means the protein RgpB that can hydrolyze peptide bonds including after the arginine in the substrate Boc-Phe-Ser-Arg-MCA; and the term “active Kgp” means the protein Kgp that can hydrolyze peptide bonds including after the lysine in Z-His-Glu-Lys-MCA.

The abbreviation “Ki” means inhibition constant. Ki can be measured as follows. Fifty microliters of an enzyme (1 nM in 50 mM bis-Tris propane [pH 8.0] containing 1% [vol/vol] Triton X-100 and 5 mM 2-mercaptoethanol) is added to columns 1 to 11 of a 96-well plate, and 100 μl was added to column 12. Two microliters a the therapeutically effective agent (100 μl in 100% DMSO) is added to column 12, the sample is mixed three times by pipetting. Then, a doubling dilution is prepared across the plate by serial transfer into adjacent wells. Fifty microliters of succinyl-Ala-Phe-Lys-(7-amido-4-methylcoumarin) (40 μM in buffer) is added to all wells, and the contents are mixed. The reaction is monitored for AMC fluorescence for 15 min at 25° C., and the progress curves are automatically converted to rates by the Fluoroskan Ascent software. This method can be used to assay, e.g., Kgp, RgpB, RgpA, trypsin, and cathepsin L. for RgpA and RgpB the substrate could be Z-Arg-AMC, for trypsin the buffer could be 10 mM Tris-10 mM CaCl₂ (pH 8.0) and the substrate could be Z-Gly-Gly-Arg-AMC, and for cathepsin L the buffer could be 50 mM sodium phosphate-1 mM EDTA (pH 6.25)-10 mM 2-mercaptoethanol and the substrate could be Z-Arg-Arg-AMC. Trypsin (human, iodination grade). The inhibition constants can then be calculated by using the following equation, with an assumption that inhibition is fully competitive:

Vᵢ=(Vₘₐₓ[S])/([S]+Kₘ(1+[I]/Kᵢ)

where Vᵢ is the observed residual activity, [S] is the substrate concentration used in the assay, Vₘₐₓ is the maximal velocity at an inhibitor concentration of zero, Kᵢ is the inhibitor dissociation constant, and [I] is the inhibitor concentration. Curves then can be fitted by nonlinear regression analysis by using fixed values for the substrate concentration and the value of the Michaelis constant (Kₘ). Data analysis can be carried out by using Prism v 2.01 (GraphPad, San Diego, Calif.).

The term “MIC₅₀” means minimum effective concentration required to inhibit the growth of 50% of the organisms.

The term “gingipains” means RgpA, RgpB, Kgp and combinations thereof.

A “therapeutically effective amount” in the present invention may vary according to factors such as the disease state, age, sex, and weight of the individual, and the ability of the agent to elicit a desired response in the individual. Dosage regimens may be adjusted to provide the optimum therapeutic response. A therapeutically effective amount is also one in which any toxic or detrimental effects of the modulator are outweighed by the therapeutically beneficial effects. The therapeutically effective amount can be determined by one of ordinary skill in the art without undue experimentation in view of the information provided in the present specification and knowledge available in the art. The experimentation required to determine the therapeutically effective amount is routine in view of the information contained in the present specification.

As used in connection with the compounds of formula I, II, III, and IV of the present invention, the terms used herein having following meaning:

“A protective group” as used in the present specification is not specifically limited as long as it does not adversely affect living organisms and synthetic reactions. It includes any group which may be suitable to protect an element or group of interest (e.g., oxygen, nitrogen, amino, guaninidino, hydrazino, etc.) from taking part in the reaction, and which may be removed after the reaction. Examples of protecting groups (e.g., amino protective groups) are described in T. W. Greene, “Protective groups in Organic Synthesis”, A Wiley-Interscience Publication, John-Wiley & Sons, New York, 1981, pp. 218-287, herein incorporated by reference. Protective groups and amino protective groups include, e.g., nitro, tert-butyloxycarbonyl (Boc), 9-fluorenylmethyloxycarbonyl (Fmoc), Alloc, tosyl, benzenesulfonyl, trifluoromethylcarbonyl, 2,2,2-trichloroethoxycarbonyl (TroC), aralkyloxycarbonyl, alkyloxycarbonyl, sulfonyl, acetyl, benzyl, 1-adamantyloxycarbonyl, aralkyloxycarbonyl, cyclopentyloxycarbonyl, which could be unsubstituted or substituted with, e.g., one or more of the following halogen(s), sulfa, amino, nitro, alkyl, aryl. Examples of aralkyloxycarbonyl groups include benzyloxycarbonyl (Cbz); benzyloxycarbonyl substituted with 1 to 3 C₁₋₄ lower alkoxy groups, e.g., p-methoxybenzyloxycarbonyl and p-ethoxybenzyloxycarbonyl; benzyloxycarbonyl substituted with a nitro group, e.g., p-nitrobenzyloxycarbonyl; benzyloxycarbonyl substituted with 1 to 3 halogen atoms, e.g., p-bromobenzyloxycarbonyl and 2,4-dichlorobenzyloxycarbonyl; diphenylmethoxycarbonyl. Examples of optionally substituted lower alkyloxycarbonyl groups include C₂₋₇ straight or branched chain lower alkyloxycarbonyl optionally substituted with 1 to 3 halogen atoms, e.g., methoxycarbonyl, ethoxycarbonyl, t-butoxycarbonyl (Boc), 9-fluorenylmethyloxycarbonyl and 2,2,2-trichloroethyloxycarbonyl.

Examples of the substituted sulfonyl group include sulfonyl having one substituent, e.g., a C₁₋₆ straight or branched chain lower alkyl group or a phenyl group optionally substituted with 1 to 3 C₁₋₆ straight or branched chain lower alkyl groups, e.g., benzenesulfonyl, p-toluenesulfonyl and methanesulfonyl. Examples of aralkyloxycarbonyl group or an optionally substituted lower alkyloxycarbonyl group include optionally lower alkoxy-, nitro- or halogen-substituted benzyloxycarbonyl, and optionally halogen-substituted C₂₋₇ straight or branched chain lower alkyloxycarbonyl (e.g., benzyloxycarbonyl or 2,2,2-trichloroethyloxycarbonyl).

Examples of substituted aralkyloxycarbonyl groups include benzyloxycarbonyl (Cbz); benzyloxycarbonyl substituted with 1 to 3 C₁₋₄ lower alkoxy groups, e.g., p-methoxybenzyloxycarbonyl and p-ethoxybenzyloxycarbonyl; benzyloxycarbonyl substituted with 1 to 3 nitro group, e.g., p-nitrobenzyloxycarbonyl; benzyloxycarbonyl substituted with 1 to 3 halogen atoms, e.g., p-bromobenzyloxycarbonyl and 2,4-dichlorobenzyloxycarbonyl; diphenylmethoxycarbonyl, etc.

Examples of substituted lower alkyloxycarbonyl groups include C₂₋₇ straight or branched chain lower alkyloxycarbonyl which may or may not be substituted with 1 to 3 halogen atoms, e.g., methoxycarbonyl, ethoxycarbonyl, t-butoxycarbonyl (Boc), 9-fluorenylmethyloxycarbonyl and 2,2,2-trichloroethyloxycarbonyl, benzyloxycarbonyl which may or may not have 1 or 2 substituents selected from the group consisting of lower alkoxy, nitro and halogen.

Examples of the lower alkoxy group include C₁₋₄, C_{1-7 or} C₁₋₆ straight-chain or branched-chain lower alkoxy, e.g., methoxy, ethoxy, n-propoxy, isopropoxy, n-butoxy, isobutoxy, t-butoxy, n-pentyloxy, isopentyloxy, n-hexyloxy and isohexyloxy, t-butoxy, C_{1-7 or} C₁₋₆ straight-chain or branched-chain lower alkyl groups having amino or C₂₋₅ lower alkoxycarbonylamino as a substituent.

Examples of the substituted piperazinyl group include piperazinyl; piperazinyl substituted with C₁₋₄ straight-chain or branched-chain lower alkyl, e.g., as N-methylpiperazinyl, N-ethylpiperazinyl and N-t-butylpiperazinyl; piperazinyl substituted with C₂₋₅ straight-chain or branched-chain lower alkoxycarbonyl, e.g., N-methoxycarbonylpiperazinyl, N-ethoxycarbonylpiperazinyl and N-t-butoxycarbonylpiperazinyl; N-benzyloxycarbonylpiperazinyl.

Examples of the optionally substituted lower alkyl group include, e.g., C_{1-7 or} C₁₋₆ straight-chain or branched-chain lower alkyl groups optionally having amino or C₂₋₅ lower alkoxycarbonylamino as a substituent. Specific examples include methyl, ethyl, n-propyl, isopropyl, n-butyl, isobutyl, t-butyl, hexyl, aminoethyl, aminopropyl, aminobutyl, methoxycarbonylaminomethyl, ethoxycarbonylaminoethyl and t-butoxycarbonylaminoethyl.

Examples of the amino group optionally substituted with lower alkyl(s) or aryl(s) include amino, methylamino, dimethylamino, ethylamino, diethylamino, n-propylamino, isopropylamino, n-butylamino and isobutylamino and like amino having 1 or 2 C₁₋₄ straight-chain or branched-chain lower alkyl groups; phenylamino, N-methyl-N-phenylamino, N-ethyl-N-phenylamino, N,N-diphenylamino, naphthylamino.

Examples of the substituted sulfonyl group include sulfonyl having one substituent, e.g., a C₁₋₆ straight or branched chain lower alkyl group, a phenyl group optionally substituted with 1 to 3 C₁₋₆ straight or branched chain lower alkyl groups, e.g., benzenesulfonyl, p-toluenesulfonyl and methanesulfonyl.

Examples of the amino protecting group include substituted aralkyloxycarbonyl group or an optionally substituted lower alkyloxycarbonyl group, e.g., a lower alkoxy-, nitro- or halogen-substituted benzyloxycarbonyl, or optionally halogen-substituted C₂₋₇ straight or branched chain lower alkyloxycarbonyl, and 2,2,2-trichloroethyloxycarbonyl.

Examples of lower alkoxy groups include C₁₋₄, C₁₋₇ or C₁₋₆ straight or branched chain lower alkoxy groups, e.g., methoxy, ethoxy, n-propoxy, isopropoxy, n-butoxy, isobutoxy, t-butoxy, etc.

“A side chain of the α-amino acid” means a side chain or residue attached to the α-carbon atom of an α-amino acid. For example, the side chain for glycine is hydrogen; for alanine it is methyl; and for valine it is isopropyl. The α-amino acid side chain can be any known naturally occurring α-amino acid side chain. Such side chains include, for example, hydrogen (for glycine), methyl (for alanine), isopropyl (for valine), n-butyl (norleucine), isobutyl (for leucine), 1-methylpropyl (for isoleucine), hydroxymethyl (for serine), 1-hydroxyethyl (for threonine), mercaptomethyl (for cysteine), 2-methylthioethyl (for methionine), carbamoylmethyl (for asparagine), carboxymethyl (for aspartic acid), 2-carboxyethyl (for glutamic acid), 2-carbamoylethyl (for glutamine), 4-aminobutyl (for lysine), benzyl (for phenylalanine) and 4-hydroxybenzyl (for thyrosin).

The side chain of lysine means the side chain or residue bonded to the α-carbon atom of lysine, i.e., 4-aminobutyl. The amino group of the side chain may be protected with a protecting group. Examples of useful amino protecting groups are the above-mentioned amino protecting groups and include a C₂₋₇ straight or branched chain lower alkyloxycarbonyl groups which may or may not be substituted with 1 to 3 halogen atoms, unsubstituted C₂₋₇ straight or branched chain lower alkyloxycarbonyl groups, t-butoxycarbonyl, etc.

The protective group for carboxyl on the above side chain is not specifically limited as long as it is a conventional protective group known to form an ester or an ether with a carboxyl group. Examples include C₁₋₆ straight-chain or branched-chain substituted or unsubstituted lower alkyl groups, e.g., methyl, ethyl, propyl, butyl, t-butyl, hexyl and trichloroethyl; substituted or unsubstituted aralkyl groups, e.g., benzyl, p-nitrobenzyl, p-methoxybenzyl and diphenylmethyl; acyloxyalkyl groups, e.g., acetoxymethyl, acetoxyethyl, propionyloxyethyl, pivaloyloxypropyl, benzoyloxymethyl, benzoyloxyethyl, benzylcarbonyloxymethyl and cyclohexylcarbonyloxymethyl; alkoxyalkyl groups, e.g., methoxymethyl, ethoxymethyl and benzyloxymethyl; and other groups, e.g., tetrahydropyranyl, dimethylaminoethyl, dimethylchlorosilyl and trichlorosilyl.

The side chain of arginine means the side chain or residue bonded to the α-carbon atom of arginine, i.e., 3-guanidinopropyl. The guanidino group of the side chain may be protected with a protecting group. The protecting group is not specifically limited as long as it does not adversely affect living organisms and synthetic reactions. Commonly used guanidino protecting groups are described, e.g., in T. W. Greene, “Protective groups in Organic Synthesis”, A Wiley-Interscience Publication, John-Wiley & Sons, New York, 1981, pp. 218-287, herein incorporated by reference. Specific examples include nitro; sulfonyl substituted with one substituent, e.g., phenyl optionally substituted with 1 to 3 C₁₋₆ straight or branched chain lower alkyl groups, or chromane optionally substituted with 1 to 6 C₁₋₆ straight or branched chain lower alkyl groups, e.g., p-toluenesulfonyl and 2,2,5,7,8-pentamethylchromane-6-sulfonyl; and oxycarbonyl groups substituted with one substituent, e.g., as aralkyl or adamantyl, e.g., benzyloxycarbonyl, phenethyloxycarbonyl and 1-adamantyloxycarbonyl. In certain embodiment, nitro is preferred.

Examples of lower alkyl groups include C₁-C₇ straight or branched chain lower alkyl groups, e.g., methyl, ethyl, n-propyl, isopropyl, n-butyl, isobutyl, s-butyl, t-butyl, n-pentyl, isopentyl, 1-ethylpropyl, n-hexyl, isohexyl, 1-ethylbutyl and the like. Preferable are methyl and ethyl.

Examples of aralalkyl groups include phenyl C₁-C₆ alkyl, e.g., benzyl and phenethyl.

“(C₁-C₇)” means a straight chain or branched non-cyclic hydrocarbon having 1, 2, 3, 4, 5, 6, or 7 carbon atoms. Representative straight chain —(C₁-C₇)alkyls include -methyl, -ethyl, -n-propyl, -n-butyl, -n-pentyl, -n-hexyl, and -n-heptyl. A branched alkyl means that one or more straight chain —(C1-C5)alkyl groups, e.g., methyl, ethyl or propyl, replace one or both hydrogens in one or more —CH2- groups of a straight chain alkyl. The total number of C atoms in a branched chain alkyl is from 3 to 7 C atoms. Representative branched —(C₁-C₇)alkyls include -iso-propyl, -sec-butyl, -iso-butyl, -tert-butyl, -iso-pentyl, -neopentyl, 1-methylbutyl, 2-methylbutyl, 3-methylbutyl, 1,1-dimethylpropyl, 1,2-dimethylpropyl, 1-methylpentyl, 2-methylpentyl, 3-methylpentyl, 4-methylpentyl, 1-ethylbutyl, 2-ethylbutyl, 3-ethylbutyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-methylhexyl, 2-methylhexyl, 3-methylhexyl, 4-methylhexyl, 5-methylhexyl, 1,2-dimethylpentyl, and 1,3-dimethylpentyl.

The term “a side chain of the α-amino acid” encompasses, e.g., side chains of lysine and arginine.

The term “halogen” includes, e.g., fluorine (F), chlorine (Cl), bromine (Br), iodine (I), and astatine (At).

The term “pharmaceutically acceptable salt” encompasses hydrates and solvates of the pharmaceutically acceptable salts. Examples of pharmaceutically acceptable salts include acid addition salts formed by reacting the compounds of the invention with pharmaceutically acceptable acids. Specific examples include inorganic acid salts, e.g., hydrochlorides and sulfates; and organic acid salts, e.g., formates, trifluoroacetates, acetates, tartrates, maleates, fumarates, succinates and methanesulfonates. The compounds of the invention or pharmaceutically acceptable salts thereof may be in the form of solvates, e.g., hydrates.

The compounds of the invention and methods of the invention encompass the use of therapeutically active enantiomers or diastereoisomers of the described compounds. All such enantiomers and diastereoisomers are included in the scope of the invention. Such compounds can be used as is as racemic mixtures or as optically resolved enantiomers or diastereoisomers.

Agents that inhibit/interfere with formation of biofilm of anaerobic gram negative bacteria include, e.g., oxantel, morantel or thiabendazole and other agents described in International Publication No. WO 2009/006699, hereby incorporated by reference.

### BRIEF DESCRIPTION OF THE DRAWINGS

FIG. 1: Examples of A) RgpB, B) Kgp, and C) RgpA staining in the Alzheimer's hippocampus compared to control hippocampus.

FIG. 2: Example of gingipain staining localization with pathology including beta amyloid plaques, alpha synuclein deposits, phospho tau, and neurodegeneration.

FIG. 3: Summary of results of findings of gingipains in postmortem brains from patients with brain disorders compared to control brains.

FIG. 4: Peripheral tissues from a patient with late onset Alzheimer's disease (Case #3) showed positive staining for gingipains. The following peripheral tissues stained positive for both Rgp and Kgp: right coronary artery, left anterior descending coronary artery, pancreas, peripheral nerve, skeletal muscle, and skin.

FIG. 5: Example of perivascular immunohistochemical staining of gingipains in human brain indicating potential point of entry.

FIG. 6: Chemical structures of certain gingipain inhibitors

FIG. 7: Intrahippocampal injection of gingipains into mouse brain causes neurodegeneration after 7 days.

FIG. 8: Differentiated neuronal SH-SY5Y cells show A) a dose dependent cell death in response to exposure to RgpB and Kgp. Iodoacetamide (IAM) which destroys the catalytic activity of the proteins prevents this effect. B) Kyt-1 and Kyt-36 can protect neurons individually and together. C) Antibodies 18E6 and 7B9 provide protection against gingipain induced cell death.

FIG. 9: BalbC mice were infected with P. gingivalis orally for 6 weeks. RgpB brain infiltration overlaps with neurodegeneration of the subgranular zone in the hippocampus.

FIG. 10: Wild Type mice infected with P. gingivalis show cognitive impairment on the Novel Object Recognition task at the 6 week time point. A) Infected mice spend equal amounts of time exploring a novel and familiar object, while normal mice spend increased time on the novel object. B) Discrimination index (T_{Novel}−T_{familiar})/Tₜₒₜₐₗ

FIG. 11: DAB-1 mice were infected twice with P. gingivalis using the chamber method. The time gap between the first and second infection was four weeks. The RgpB brain infiltration overlaps with neurodegeneration of the CA3/CA2 granular cell zone of the hippocampus.

FIG. 12: Pharmacokinetic data of Kyt-36 intravenous, subcutaneous, oral and oral+Ritonavir. Ritonavir increases the half-life of oral Kyt-36.

FIG. 13: Aged dogs with cognitive impairment all strongly positive for brain Kgp. Young dog hippocampus and entorhinal cortex are negative for Kgp.

### DETAILED DESCRIPTION OF THE INVENTION

Porphyromonas gingivalis (P. gingivalis) is an anaerobic gram-negative rod. P. gingivalis produces several extracellular proteases, including, e.g., Arginine Gingipain A (RgpA), Arginine Gingipain B (RgpB) and Lysine Gingipain (Kgp). These proteases may degrade a broad range of proteins of connective tissue and plasma (e.g., immunoglobulins, proteinase inhibitors and collagen, etc.), get into systemic circulation and/or enter the brain. These proteases may also cause disruption to kallikrein—kinin cascade, blood coagulation, and the host defense systems.

Recently, lipopolysaccharide (LPS) from the periodontal pathogen P. gingivalis was discovered in 4 out of 10 AD brains[20]. The same study, utilizing two different antibodies, failed to localize one of the three P. gingivalis associated cysteine proteases, Arginine gingipain A, by immunohistochemistry in any of the Alzheimer's brains. It is possible that the antibodies used were inactive or too dilute as no positive controls were described. A study published as an abstract at the Alzheimer's Association International Conference in 2013 found that oral infection of transgenic APP mice (J20 mice) with P. gingivalis triggers abeta plaque formation (a hallmark of Alzheimer's disease), neuroinflammation and cognitive impairment [21]. The authors concluded that inflammation may be a target for therapy. A study by Poole et al. found that P. gingivalis DNA could be found in the brains of 50% of ApoE−/− mice after oral infection[22] This indicates that the bacteria can infiltrate the brain.

As described herein, through human and dog brain immunohistochemistry studies, all three P. gingivalis expressed cysteine proteases, known as Arginine Gingipain A (RgpA) Arginine Gingipain B (RgpB) and Lysine Gingipain (Kgp), can infiltrate the human brain, and depending on the amount and location of this infiltration, can cause or contribute to various brain disorders. The gingipains contribute to many functions of the organism including its survival and virulence.

RgpB is a lower molecular weight form of RgpA[23]. The RgpB-specific monoclonal antibody, 18E6, is specific for the first half of the immunoglobulin domain of RgpB[17]. The Kgp-specific monoclonal antibody, 7B9, recognizes an epitope within the Kgp catalytic domain[18].

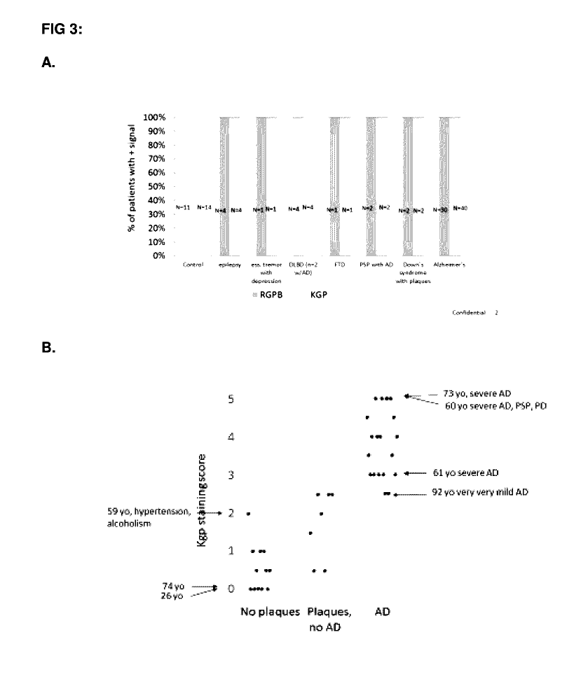

FIG. 3 presents a summary of human autopsy immunohistochemistry findings. Kgp has been positively identified in brain samples from patients with neurological disorders as follows: 40/40 Alzheimer's disease patients, 10/10 reportedly presymptomatic patients with Alzheimer's pathology (abeta plaques), 1/1 progressive supranuclear palsy patient with co-morbid Alzhemier's, 1/1 frontal temporal dementia patient, 1/1 Parkinson's patient with co-morbid Alzheimer's and Lewy body disease, 4/4 patients with dementia with Lewy body disease (2 with co-morbid Alzheimer's), 1/1 essential tremor patient with depression, 2/2 Down's syndrome patients 33-34 years of age, 4/4 epilepsy patients. In reportedly healthy controls: 5/13 brains were negative for staining, 4/13 brains have trace staining, and the remaining 4/13 had Kgp levels higher than trace levels, but lower than cases with brain disease (FIG. 3).

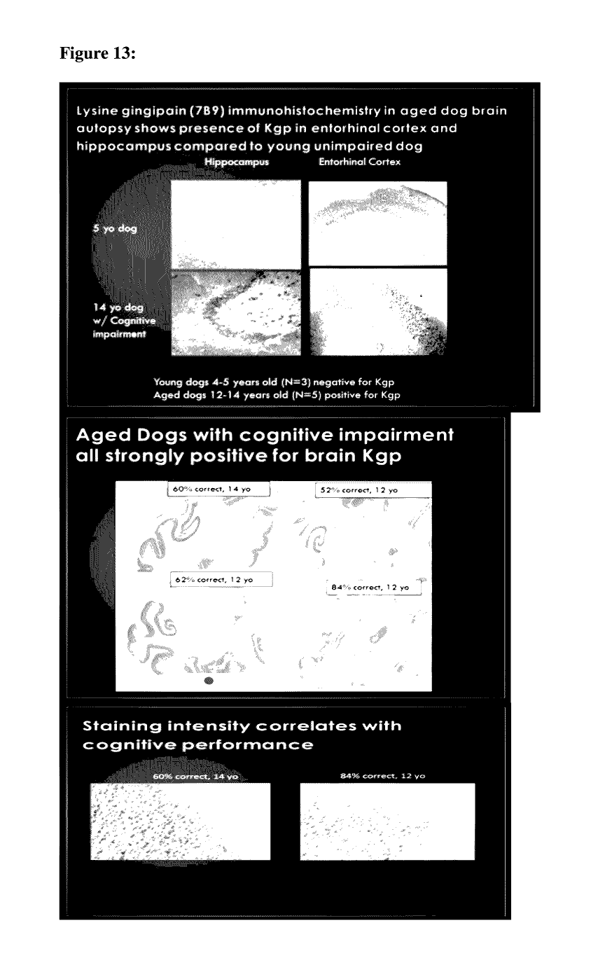

In addition, Kgp was positively identified in brain samples from five beagle dogs with canine cognitive disorder (CCD) (FIG. 13) but it was absent from 3 young cognitively normal dogs

Gingipains can be secreted, transported to outer membrane surfaces of Porphyromonas gingivalis, or released in outer membrane vesicles by the bacterium[24]. Porphyromonas gingivalis has previously been identified in periodontal tissues[25], coronary arteries[26], aorta[27], and recently, the liver[28]. Release of gingipains from any of these niches into the systemic circulation could result in translocation of gingipains to the brain or retina.

Alzheimer's is associated with retinal degeneration[29] and it has been proposed that age-related macular degeneration (AMD) is an “Alzheimer's disease of the eye” [30]. The retina is composed of light sensitive neurons (photoreceptors) which transmit information through several neuronal layers to the brain via the optic nerve. The retina is usually protected by the blood ocular barrier, similar to the blood brain barrier, which could be breached by P. gingivalis due to aging, inflammation or progressive gingipain induced degeneration[31]. AMD and glaucoma share a number of clinical and pathological features with Alzheimer's disease [32] including inflammation, progressive neuronal degeneration, activation of the immunological complement pathway [33] and protein deposits. Aging, hypercholesterolaemia, hypertension, obesity, arteriosclerosis, and smoking are risk factors to for both AMD and AD. Oral inflammatory infections have been associated with AMD[5].

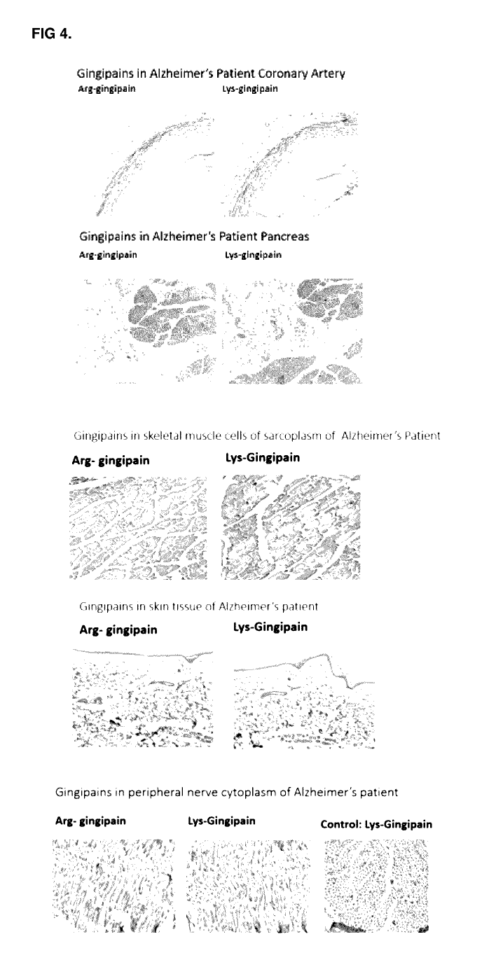

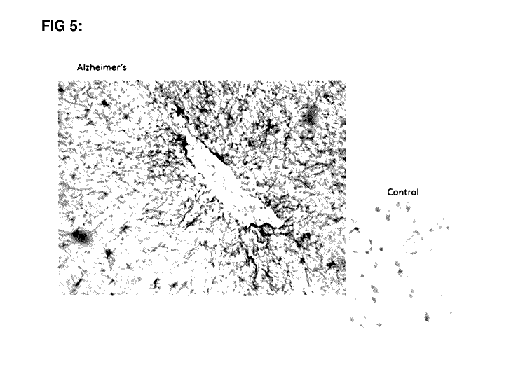

In the present invention, evidence is provided from an 89-year-old Alzheimer's subject that gingipains may be widely dispersed throughout the body, as gingipains were identified not only in brain tissue, but also in tissue from coronary arteries, pancreas, peripheral nerve, skeletal muscle, and skin (FIG. 4). Localization in these tissue can contribute to the peripheral disorders discussed herein. While not wanting to be bound to a particular theory, the identification of gingipains deep in the media of coronary blood vessels as seen in FIG. 4, could indicate a mechanism by which gingipains can burrow through blood vessels in the brain and breach the blood-brain barrier. In support of this concept, immunohistochemical evidence was found of perivascular staining of gingipains around blood vessels in the brain, with a pattern of gingipain staining radiating out from penetrating vessels (FIG. 5).

Gingipains may enter the brain by degrading endothelial cells protecting the blood brain barrier, or by a traumatic event such as a stroke or traumatic brain injury, which permanently or transiently reduces the integrity of the blood brain barrier. Such a disruption, in traumatic brain injury, for example, may contribute to the infiltration of gingipains in infected individuals and subsequent development of chronic neurodegenerative conditions like chronic traumatic encephalopathy (CTE) in athletes and military personnel. People who are at a high risk of traumatic injury or stroke could be preventatively treated with gingipain inhibitors to reduce the risk of CTE or stroke induced pathology.

Gingipains may also reach the brain through other mechanisms including active transport, passive transport or macrophage delivery.

Patients with gingipains in their brain and circulatory system may experience mild cognitive impairment, age associated memory impairments or generalized accelerated aging due to gingipain induced cell death which could be treated or prevented with compounds that inhibit gingipains, including the compounds described above.

Such “accelerated aging” may be limited to the brain or may be experienced in a variety of tissue types accessible to the circulatory system. Similarly, dogs with gingipains in their brain and circulatory system may experience mild cognitive impairment, age associated memory impairments or generalized accelerated aging due to gingipain induced cell death, which could be treated or prevented with compounds and compositions that inhibit gingipains (e.g., Kyt-41, Kyt-1, Kyt-36, compounds of Formulas I, II, III, or IV, and other compounds described above) or act as bacteriocidal or bacteriostatic agents of P. Gingivalis. For example, it has been reported that tetracyclines at 100 μM totally inhibited the amidolytic activity of arginine-specific gingipains (HRgpA and RgpB). Among tetracycline derivatives, the most potent gingipain inhibitor was doxycycline, followed by tetracycline and minocycline. RgpB was inhibited by doxycycline in an uncompetitive and reversible manner with a 50% inhibitory concentration of 3 μM. Imamura, et al.; Inhibition of Trypsin-Like Cystein Proteinases (Gingipains) from Porphyromonas ginivalis by Tetracycline and its Analogues, October 2001 vol. 45 no. 10 2 871-2876. Stathopoulou et al. found that small peptide derived inhibitors of Rgp or Kgp can prevent gingipain induced epithelial cell death.[34] Rgp and Kgp induced cell death has also been demonstrated in endothelial cells and other cell types.[35, 36]

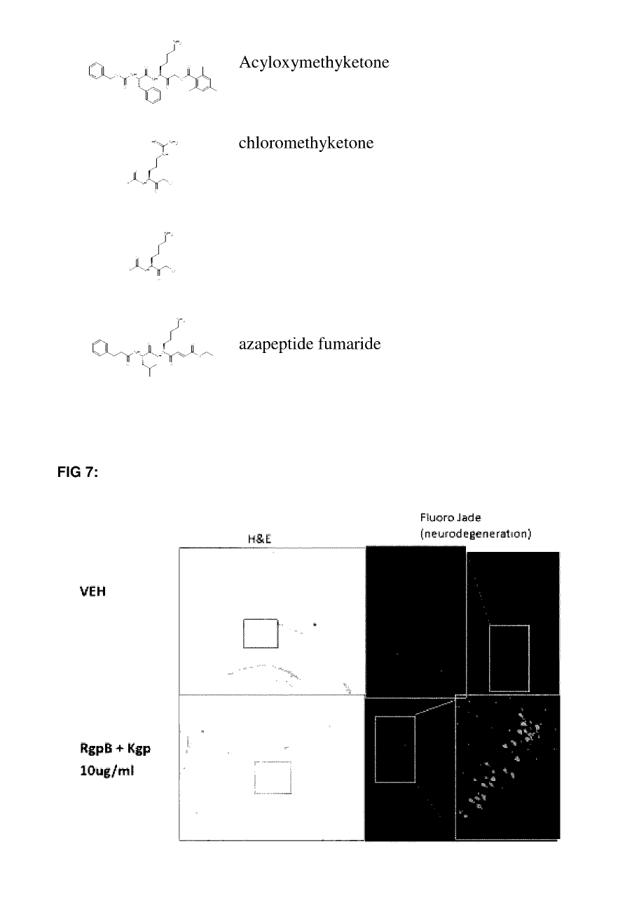

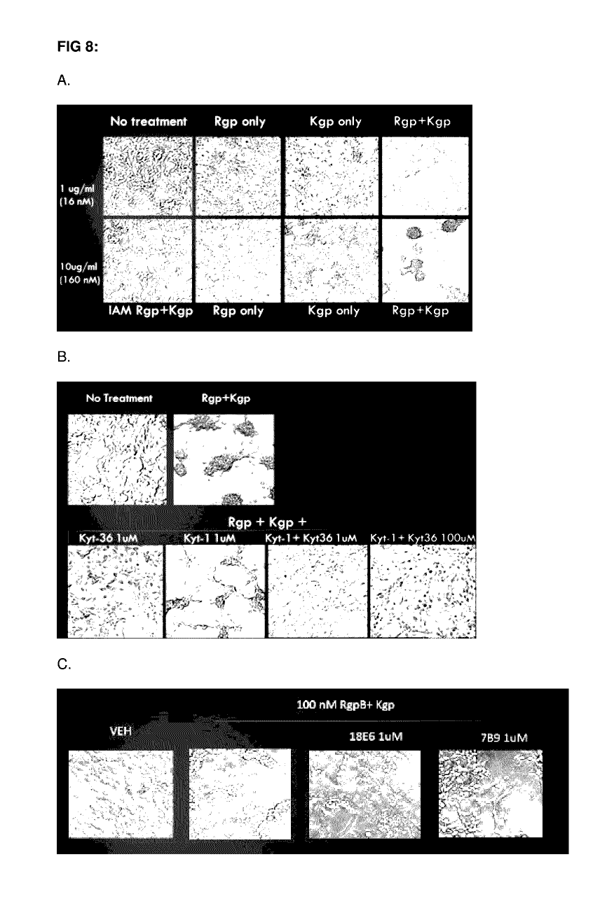

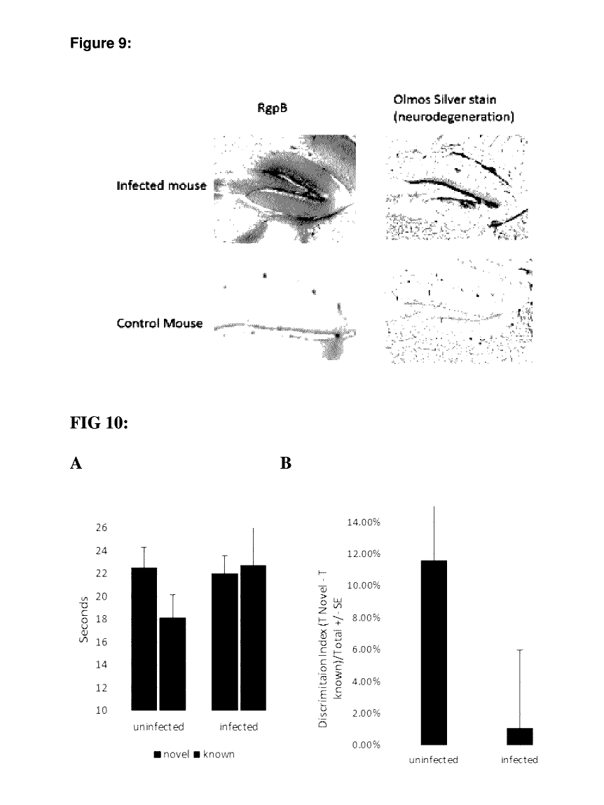

As demonstrated herein, exposure of differentiated SH-SY5Y neuronal cells to gingipains caused dose dependent cell death within 24 hours (FIG. 8). It was shown for the first time that a Kgp inhibitor has a stronger neurotoxic effect on neurons than an Rgp inhibitor, and that Kgp inhibitors (e.g.: Kyt-36) are more protective than Rgp inhibitors. Knock out of Kgp from P. gingivalis has a more potent effect on bacterial growth inhibition than knock out of RgpB and resulted in a synergistic 50% reduction of RgpB activity after 6 days potentially due to reduced export [37]. Intrahippocampal injection into the mouse brain of gingipains resulted in visible neurodegeneration within 7 days (FIG. 7). Oral infection of BALB/C mice resulted in gingipain infiltration and correlating neurodegeneration after 6 weeks (FIG. 9) as well as cognitive impairment on the novel object recognition task (FIG. 10). Infection of DBA-1 mice with Porphyromonas gingivalis using the chamber model[38] also resulted in brain gingipain infiltration and correlating neurodegeneration after two bacterial injections to the subcutaneous chamber separated by 4 weeks (FIG. 11). The presence of these proteases in the brain and their proven capability to produce cell death indicates that they play a role in neurodegeneration.

One embodiment of the invention encompasses the use of P. gingivalis exposed or infected rodent and dog as models of brain disorders. These models are valuable for the understanding of brain disorders and the testing of therapeutics for efficacy.

As described previously, dogs are naturally predisposed to infection with P. gingivalis and incidence of this infection is increased through direct exposure, feeding of soft food, and/or lack of dental care.[14, 15] Aged dogs spontaneously develop many features of AD including cognitive decline and brain neuropathology[13]. Analysis of the brains of aged dogs with cognitive impairment and pathology show the presence of brain lysine gingipains, while younger dogs do not have brain gingipain (FIG. 13). One embodiment of the invention encompasses the use of gingipain inhibitors to treat or prevent brain disorders in dogs. Additionally, dogs with natural or purposeful P. gingivalis infection can be useful as models for studying the efficacy of therapeutics for humans.

Bacteriocidal and bacteriostatic compounds like antibiotics can be used to reduce bacterial levels and resulting gingipain and inflammation induced neurodegeneration. Preferably an antibiotic used for treatment of brain disorders would have selectivity for P. gingivalis over other bacteria so as to preserve beneficial bacteria. Candidates include but are not limited to quinolones (e.g., gemifloxacin, ciprofloxacin, oflaxacin, trovafloxacin, sitafloxacin, etc.), β-lactams (e.g., a penicillin (e.g., amoxicillin, amoxacilin-clavulanate, piperacillin-tazobactam, penicillin G, etc.), cephalosporins (e.g., ceftriaxone, etc.)), macrolides (e.g., erythromycin, azithromycin, clarithromycin, etc.), carbapenems (e.g., doripenem, imipenem, meropinem, ertapenem, etc.), thiazolides (e.g, tizoxanidine, nitazoxanidine, RM 4807, RM 4809, etc.), tetracyclines (e.g., tetracycline, minocycline, doxycycline, eravacycline, etc.), clindamycin, metronidazole, satranidazole, agents that inhibit/interfere with formation of biofilm of anaerobic gram negative bacteria (e.g., oxantel, morantel or thiabendazole, etc), combinations of two, three, four, five, six, or more of any of the foregoing agents, etc. In certain embodiments, the compound is selected from the group consisting of tetracyclines (e.g., minocycline, doxycycline, etc.), penicillins (e.g., amoxicillin/clavulanic acid combinations), metronidazole, eravacycline, clindamycin, amoxicillin, eravacycline, satranidazole, and combinations of the foregoing.

FA-70C1, isolated from the culture supernatant of Streptomyces species strain FA-70, is a potent Rgp inhibitor and also exhibits growth-inhibitory activity against P. gingivalis (REF).

Experiments have shown that the dipeptide bestatin selectively inhibits growth of P. gingivalis by affecting the intracellular uptake of amino acids and peptides, which serve as energy and nitrogen sources for this bacterial species (Labbe et al, 2001). Bestatin or similar compounds could be used to treat brain disorders, diabetes, arthritis, cardiovascular disease, or pneumonia.

Alternatively, a treatment can be targeted to inhibit the gingipains in order to simultaneously block gingipain induced cell death and act as a narrow spectrum antibiotic.

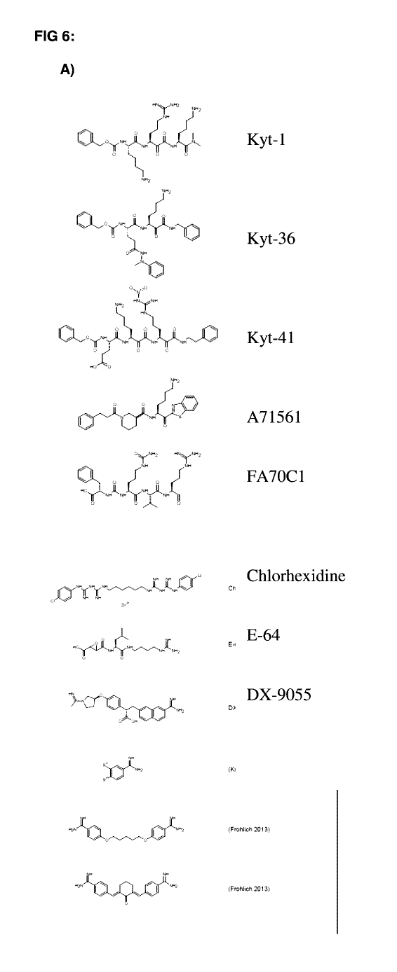

Several series of compounds, in particular, have been identified with the ability to inhibit gingipains. FIG. 6 provides structures of some of the compounds. Kyt-41 was found to be a potent and specific dual inhibitor of Kgp and RgpB that was found to be effective in treating periodontal symptoms in dogs²⁶.

The present invention encompasses the use of compounds described herein to treat or prevent a variety of brain disorders including, but not limited, to Alzheimer's, stroke, Parkinson's, Lewy body disease, epilepsy, chronic traumatic encephalopathy, depression, schizophrenia, autism, multiple system atrophy and others. It is conceivable that any number of brain disorders could be caused by the infiltration of gingipains into the brain, depending on the site and amount of infiltration.

Based on data published by Kadowaki et al. and Katoaka et al. it is believed that Kyt-41, Kyt-1 or Kyt-36, or other compounds in the series might be used alone or in combination to be effective[39]′²⁶. Kyt-1 and Kyt-36 have been tested in a cell model of gingipain neuronal toxicity. Kyt 36 alone, and Kyt-1 together with Kyt-36 prevented gingipain induced neurodegeneration (FIG. 8). Further, other unrelated therapeutic agents can be administered in combination with the Kyt or related compounds.

Additionally, the present invention encompasses various administration modes by which the compounds might be delivered to increase bioavailability or blood brain barrier penetration, not previously conceived for the compounds, including but not limited to, intravenous, intranasal, intrathecal, subcutaneous, intracranial, buccal and oral.

The invention also encompasses pharmaceutically acceptable compositions, formulations or dosage forms comprising one or more pharmaceutically active compound(s) described herein and one or more pharmaceutically acceptable excipients for treatment of a brain disorder (e.g., a neurodegenerative disease (e.g., Alzheimer's disease, Down's syndrome, epilepsy, autism, Parkinson's disease, essential tremor, fronto-temporal dementia, progressive supranuclear palsy, amyotrophic lateral sclerosis, Huntington's disease, multiple sclerosis, mild cognitive impairment, age associated memory impairment, chronic traumatic encephalopathy, stroke, Lewy Body disease, multiple system atrophy, schizophrenia and depression, etc.), diabetes, cardiovascular disease, and arthritis. The pharmaceutically acceptable excipients are described in Handbook of Pharmaceutical Excipients, 4^{th} Edition, 2003, herein incorporated by reference.

For example, formulations for sustained-release (SR), sustained-action (SA), extended-release (ER, XR, XL) timed-release (TR), controlled-release (CR), modified release (MR), continuous-release, osmotic release and slow release implants. Time release technology can be used to increase bioavailability in certain embodiments. These alternative routes of administration were not previously considered for some of the disclosed compounds, the compounds which were primarily contemplated to be formulated for topical gingival delivery and not for systemic delivery.

The use of hybrid molecules to promote active transport or nanoparticles might be used to increase blood brain barrier transport in certain embodiments of the invention. For example liposomes, proteins, engineered peptide compounds or antibodies that bind to the receptors that transport proteins across the blood brain barrier including LPR-1 receptor, transferrin receptor, EGF-like growth factor or glutathione transporter may be used to increase penetration into the brain. Physical techniques including osmotic opening, ultrasound, lasers, sphenopalantine ganglion stimulation, direct intracranial, intrathecal, or intraventricular delivery via a pump might be used. Because it was unknown that gingipain inhibitors could be used to treat brain disorders, inventions related to blood brain barrier transport had not been anticipated previously.

Effective doses of the compounds or compositions of the present invention, for the treatment of the above described conditions vary depending upon many different factors, including means of administration, target site, physiological state of the patient, other medications administered, and whether treatment is prophylactic or therapeutic. Treatment dosages need to be titrated to optimize safety and efficacy. In view of the information provided in the specification and the knowledge currently available in the art, effective doses and amount could be determined by one of ordinary skill in the art without undue experimentation. The experimentation required to determine the effective doses/amounts would be routine in view of the present specification.

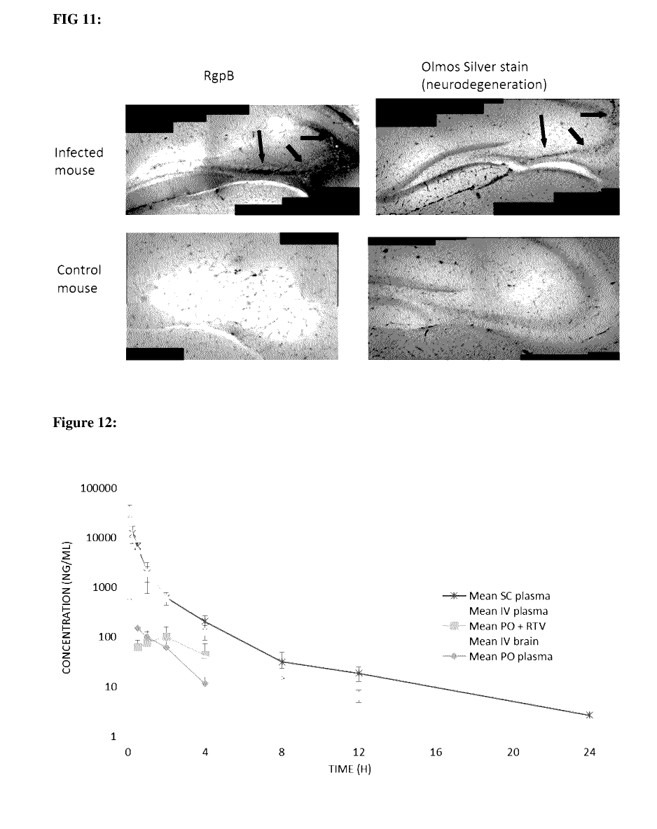

In another embodiment, “boosting” of gingipain protease inhibitor compounds with ritonavir (RTV) may be used to increase bioavailability and increase blood brain barrier penetration. For example, ritonavir is commonly combined with oral peptidic HIV protease inhibitors to increase plasma levels by inhibiting the P450 3A4 enzyme and thus decreasing first-pass metabolism[40]. In addition, RTV binds to P-glycoprotein, a transmembrane efflux pump that is found in many tissues, including the blood brain barrier, allowing co-administered compounds better access to the brain[41]. Therefore, a combination of RTV and gingipain protease inhibitors might be used to increase plasma concentrations and brain levels of the gingipain inhibitors. It is shown herein that oral administration of RTV 15 minutes prior to Kyt-36 increases the half life (FIG. 12). In another embodiment, a course of antibiotics might be paired with gingipain inhibitors to jump start treatment.

The efficacy of the administration/treatment may be accessed by measuring levels of circulating levels of RgpA and/or RgpB and/or Kgp. Based on this assessment, the dose and/or frequency of administration may be adjusted.

The efficacy of the administration/treatment may be accessed by measuring levels of circulating levels hemoglobin. Based on this assessment, the dose and/or frequency of administration may be adjusted.

In another embodiment natural gingipain inhibitors including melabaricone C, isolated from nutmeg or polyphenolic compounds derived from plants, such as cranberry, green tea, apple, and hops could be formulated for treatment or prevention of brain disorders (Tanaguchi et al. 2014) for use alone or in combination with each other or the compounds described above. Naturally and unnaturally occurring antimicrobial peptides including: κ-casein peptide (109-137) 34, histatin 5, and CL(14-25), CL(K25A) and CL(R24A, K25A) (Taniguchi et al. 2014) alone or in combination with each other or the compounds described above. he efficacy of the administration/treatment may be accessed.

In another embodiment bacteriophage therapy against P. gingivalis can be used as a treatment for brain and other disorders [42]. Phages specifically infect their target bacteria, replicate and then lyse those bacteria, releasing progeny that can continue the cycle, including migrating to other sites of infection anywhere in the body including the brain [43]. Phages only minimally impact non-target bacteria or body tissues.

Phage therapy for brain disorders may be cocktails of phages purified from oral preparations that display a wider spectrum of activity than for P. gingivalis or may be highly specific for P. gingivalis. Lytic (as opposed to temperate) phages suitable for therapeutic purposes can be isolated from oral tissue or saliva of periodontal patients by methods known to those in the art and as described in Mancuca et al. 2010. [44] Briefly, P. gingivalis and its phages can be isolated by culturing the human samples in selective Bacteroides gingivalis agar[45] or by enriching for P. gingivalis by adding culture of P. gingivalis to the sample to ampilify the specific phages. The Phage are then isolated by centrifugation followed by filtraition or chloroform step to separate phages from bacterial hosts. The phage are then applied to a lawn of bacteria and lytic plaques are selected for further characterization. Ideally multiple phage from diverse strains of P. gingivalis or even multiple types of oral bacteria would be combined for a more effective therapy. The choice of phage strain and the methods of phage preparation are critical to the success or failure of phage therapy. Insufficiently virulent phages or poorly prepared phage stocks can result in ineffective therapy[46].

In another embodiment, antibodies targeting gingipains or other P. gingivalis protein could be used as therapeutics. Antibodies may rely on damage to the blood brain barrier for access to the brain or peripheral interference with gingpains and P. gingivalis propagation. Antibodies may also help to stimulate the efficacy of the immune system in clearing the bacteria. New or existing antibodies to RgpA and/or RgpB and/or Kgp can be utilized including, e.g., 18E6 and 7B9 described above. It has been demonstrated that 18E6 and 7B9 protect cells from gingipain induced cell death (FIG. 8C). An RgpA antibody 61BG1.3 has previously demonstrated efficacy topically in prevention of recolonization by P. gingivalis after periodontal treatment [19]. Additional antibodies which may be used in the methods of present invention include, e.g., RgpA MAb 1B5, RgpA Mab 7B4, and Kgp antibody 15C8, as well as structurally and/or functionally similar antibodies.

Antibodies would preferably be humanized for use in humans.

Methods known to those in the field for delivery of biologics to improve half-life and brain penetration can be used including, but not limited to, intravenous delivery, subcutaneous delivery, intranasal delivery, intrathecal delivery, vector transport, and direct brain delivery. Treatments for the retina may be delivered directly to the eye.

Additionally, vaccines have been used to treat a disease in progress and a vaccine could be administered to a person with a brain disorder to disrupt progression. A variety of vaccine strategies are known to those in the art. These strategies could be used for the treatment and prevention of brain disorders as described in the present specification[20].

Genco et al. immunized mice with a 20 amino acid synthetic peptides derived from the catalytic domain of RgpA having the amino acid sequence YTPVEEKQNGRMIVIVAKKY [47]. Mice vaccinated with this synthetic gingipain epitope were protected from P. gingivalis invasion in the mouse chamber model. In another study, mice immunized with purified RgpA protein were protected from P. gingivalis-mediated periodontal disease, suggesting that it was the production of antibodies against the hemaglutinin domain of RgpA that were protective[48]. If one or more gingipains are used for immunization, it may be preferably to inactivate their proteolytic activity, using iodoacetamide or other method, prior to administering. Recombinant gingipains, produced by E. Coli for example, have little or no activity compared to gingipains produced by P. gingivalis and can be used for this purpose.

Page et al. have shown that immunization of nonhuman primates with purified gingipains inhibited alveolar bone destruction, and that the vaccine was well tolerated by the animals[49]. Gingipain DNA vaccines were tested by Guo et al. who found that Kgp and RgpA, but not RgpB DNA vaccines were effective in preventing periodontal symptoms in dogs [50]. The kgp, rgpA, and rgpB genes were amplified by polymerase chain reaction (PCR) from Porphyromonas gingivalis (Pg) ATCC 33277 and cloned into the pVAX1 vector.

Lee et al. 2006 immunized rats with P. gingivalis HSP60, and experimental alveolar bone loss was induced by infection with multiple periodontopathogenic bacteria. There was a very strong inverse relationship between postimmune anti-P. gingivalis HSP immunoglobulin G (IgG) levels and the amount of alveolar bone loss induced by either P. gingivalis or multiple bacterial infection (p=0.007). Polymerase chain reaction data indicated that the vaccine successfully eradicated the multiple pathogenic species.

Intranasal administration of P. gingivalis fimbrial antigen with recombinant cholera toxin B subunit also induced a significant immune response (fimbrial-specific secretory IgA-sIgA) in mice, which could reduce P. gingivalis-mediated alveolar bone loss. It is also possible that this mucosal immunization resulted in peripheral tolerance and hence a reduced inflammatory response and alveolar bone loss. However, it has further been demonstrated that immunization with 43-kDa fimbrillin polymer of P. gingivalis did not show satisfactory levels of protection against all strains of P. gingivalis tested [21]. The feasibility of this fimbrial protein of P. gingivalis as a vaccine candidate antigen may therefore be dependent on its effectiveness in protecting against all the P. gingivalis strains.

A conjugate vaccine incorporating both fimbriae and P. gingivalis capsular polysaccharide (CPS) has been introduced in a study that led to the production of a high IgG response and which was effective in protecting against P. gingivalis infection [22]. However, in this study it was not clear whether the protection came from the CPS or fimbriae. CPS, by virtue of its encapsulation and antigenic shift, constitutes a robust strategy for P. gingivalis survival against opsonophagocytic activity. Due to its poor T-cell stimulating ability, however, CPS of other Gram-negative bacteria is usually conjugated to a protein antigen (for stimulating helper T-cells) in many vaccine trials in infectious diseases, such as pneumonia and meningitis. More recently, P. gingivalis CPS alone has nevertheless, been used as an immunogen, and it has been reported to result in an elevated production of serum IgG and IgM that provided protection against P. gingivalis-induced bone loss.

Zhu et al. 2013 tested a vaccine of peptidylarginine deiminase (PAD) from P. gingivalis in a mouse model. Compared with animal immunization with incomplete Freund's adjuvant (IFA) alone, PAD group significantly inhibited (P<0.05) bone resorption. It is contemplated that these vaccine strategies could be used in the methods of the present invention, e.g., to treat or prevent Alzheimer's disease [51].

Vaccination can also be accomplished with live attenuated or killed P. gingivalis. Comparisons of the protective effects of subcutaneous immunization among formalin-killed P. gingivalis, heat-killed P. gingivalis, outer membrane fraction, and lipopolysaccharide revealed that immunization with killed P. gingivalis provides the greatest protection from lesion formation induced by P. gingivalis [52]. Preparation of killed bacteria for vaccination involves growth of the bacteria in standard conditions, and collection during the log-arithmic growth phase. The bacteria are then centrifuged, washed three times, and resuspended in sterile phosphate-buffered saline (PBS, pH 7.4). Bacteria can be killed by fixation with 0.8% formalin at 4° C. for 24 h followed by washing, and resuspension in sterile PBS or heated to 95 deg C. for 10 minutes[53, 54]. Bacteria can be plated and incubated for 7 days to ensure effective killing. Approximately, 2.5×109 cells/mL or other effective dose can be used for vaccination. Live attenuated bacteria may be the most effective vaccine for intracellular bacteria as is the case for tuberculosis. P. gingivalis may be attenuated by knocking out gingpains or other methods known to those in the art.

Vaccination in accordance with the present invention also encompasses passive immunization, namely direct administration of antibodies against gingipains. The antibodies 7B9, 18E6 or 61Bg 1.3, 1B5, 7B4, 15C8, or a structurally and/or functionally similar antibodies, could be used in the methods of the present invention, e.g., to treat or prevent Alzheimer's disease or determine if a subject possibly has Alzheimer's disease, is at risk of developing the Alzheimer's disease, as well as to monitor the efficacy of the treatment of Alzheimer's disease. Humanized versions of these antibodies, nanobodies, or antibody fragments of these antibodies could also be used. Efficacy of 7B9 and 18E6 in prevention of gingipain induced cell death is shown in FIG. 8 C.

In some of the embodiments, the passive immunization prevents or treats infection of P. gingivalis and therefore prevent brain infiltration of gingipains. Since colonization with P. gingivalis can occur during the first few years of life[55], a vaccine against gingipains or other components of Porphyromonas gingivalis may provide life-long protection against gingipain-induced neurodegeneration. Transmission of Porphyromonas gingivalis is thought to occur through intimate contact, including from an infected parent to a child[56-58]. This could explain symptoms seen in disorders such as mental retardation, autism and Down's syndrome, in addition to pediatric epilepsy.

Several therapeutics are on the market and in development for Alzheimer's disease. It is envisioned that these therapeutics can be used in combination with P. gingivalis targeted therapies (i.e., therapies using the compound(s) described above). Specifically, P. gingivalis targeted treatments can be used in fixed or variable dose combination with cholinesterase inhibitors (like donezepil), NMDA antagonists (like memantine), and/or development stage therapeutics targeting abeta, tau, ApoE or neuroinflammation. The use of the compounds of formula (I), (II), (III) and (IV) in combination with one or more of the following is also envisioned: Aβ peptides level reducers, pathogenic level tau reducers, microtubule stabilizers, agents capable or removing atherosclerotic plaques, agents that lower circulating levels of β-amyloid and tau, modulators of autophagy, neurotransmitter level regulators, GABA(A) α5 receptors inhibitors, and additional agents that help maintain and/or restore cognitive function and functional deficits of Alzheimer's disease, and/or slow down decline in cognitive functions and functional deficits in Alzheimer's disease. Combination treatments are also envisioned with treatments that promote neuroplasticity and recovery of the brain, including but not limited to growth factors, growth factor mimetics, stem cells, gene therapy and encapsulated cell therapy.

Low levels of hemoglobin and/or anemia is associated with Alzheimer's disease and may be caused by P. Gingivalis infection [59] [60]. Thus, in a further aspect, the invention is directed in part to a method of identifying a subject as possibly having or at risk of developing Alzheimer's disease comprising determining whether a subject has a low hemoglobin level, low cell hematocrit and/or an anemia. A low level hemoglobin level for an adult male is a level of below 13.5 g/dL. A low level for a non-pregnant adult woman is a level below 11.9 g/dL. A low level for a pregnant woman is a level below 10.8 g/dL. The detection of low hemoglobin level, low hematocrit, or anemia indicates that the subject possibly has or at risk of developing Alzheimer's disease or other brain or retinal disorder.

In a further aspect, the invention is directed in part to a method of treating a subject diagnosed with Alzheimer's disease comprising periodically determining a serum hemoglobin level in a subject, and adjusting and/or changing the treatment if the level of hemoglobin is increasing or decreasing. Adjusting treatment means increasing the dose of a pharmaceutically acceptable agent. Changing treatment means replacing one of the pharmaceutically acceptable agent(s) with a different pharmaceutically acceptable agent or adding an additional pharmaceutically acceptable agent.

The present invention also provides for a diagnostic based on imaging gingpains in the human brain. Any agent that binds to gingipains, including but not limited to Kyt-41, Kyt-1, Kyt-36 and other compounds in FIG. 6 and described in this invention or elsewhere, can be labeled with F18, I124 or other radiographic markers and visualized using positron emission tomography (PET) or SPECT scanning

### Example 1

FIGS. 1 & 3 present examples and a summary of human autopsy immunohistochemistry findings, respectively. Kgp has been positively identified in brain samples from patients with neurological disorders as follows: 40/40 Alzheimer's disease patients, 10/10 reportedly presymptomatic patients with Alzheimer's pathology (abeta plaques), 2/2 progressive supranuclear palsy patients with co-morbid Alzheimer's, 1/1 frontal temporal dementia patient, 1/1 Parkinson's patient with co-morbid Alzheimer's and Lewy body disease, 4/4 patients with dementia with Lewy body disease (2 with co-morbid Alzheimer's), 1/1 essential tremor patient with depression, 2/2 Down's syndrome patients with abeta plaques 33-34 years of age, 4/4 epilepsy patients. In reportedly healthy controls (age 11-74 years old): 5/13 brains were negative for staining, 4/13 brains have trace staining, and the remaining 4/13 had Kgp staining higher than trace levels, but lower than cases with brain disease (FIG. 3).

### Example 2

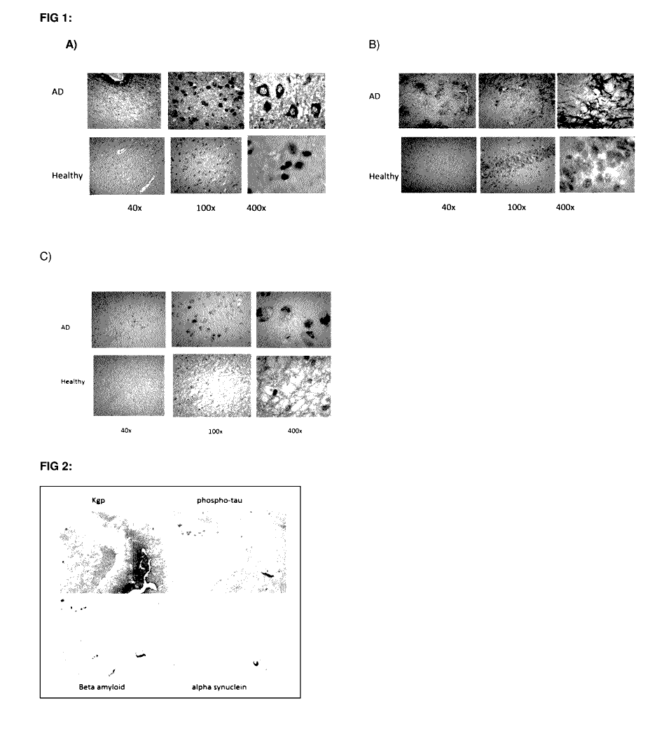

Peripheral tissue specimens including skin, pancreas, coronary artery, muscle and peripheral nerve were selected from an 89 year old Alzheimer's patient analyzed with immunohistochemistry for Kgp and RgpB. The pathology report states that the hippocampus and entorhinal cortex, contained frequent neuritic plaques and tau proteins frontal lobe cortex. Frontal Cortex contains neuritic plaques, neurofibrillary tangles, and amyloid angiopathy. CERAD Stage VI, BRAAK Stage 6, and amyloid angiopathy of small vessels in the brain. All peripheral tissues show staining for Kgp and RgpB indicating that gingipain induced cell degeneration may be responsible for co-mordities of Alzheimer's including cardiovascular disease, diabetes, arthritis, retinal disorders and peripheral nerve disorders (FIG. 2).

### Example 3

Adult male mice (CD-1, 25 g approximately) n=6 per group were anaesthetized and injected unilaterally intrahippocampally using standard stereotaxic techniques. Gingipains RgpB and Kgp, purified from P. gingivalis and provided by Dr. Jan and Barbara Potempa, were diluted prior to injection to 10 ug/ml. Seven days post-surgery the animals were anaesthetized, perfused and humanely killed and brains removed and sectioned for histological analysis. Fluoro-Jade staining was then be performed on sections of hippocampus to assess for neurodegeneration (Schmued L C and Hopkins K J, 2000). Fluoro-Jade staining identifies cell bodies, dendrites, axons and axons terminals of degenerating neurons but does not stain healthy neurons, myelin, or vascular elements.

Brain sections were examined with an epifluorescence microscope (Nikon Microphot FXA) using a filter system suitable for visualizing fluorescein or fluorescein isothiocyanate (FITC). Images were acquired with a Leica DC Camera and an Image Analysis software (Leica IM50). Fluoro-Jade C-positive degenerating neurons appeared bright yellow-green against a dark background and were clearly identified in the animal groups treated with Gingipains. No Fluoro-Jade C-positive cells were observed in vehicle-treated group. (FIG. 5).

### Example 4

SH-SY5Y neuroblastoma cells were cultured and differentiated in the presence of 5 uM retinoic acid based on established methods [62]. Differentiation into neuronal cells was verified by observation of neurite outgrowth. The differentiated cells were exposed to 100 nM Kgp and/or RgpB for 24 hours, in the presence or absence of gingipain inhibitors. Results were recorded using a digital microscope camera. An irreversible inhibitor of Kgp and RgpB, iodoacetamide (IAM), blocks toxicity from combined Kgp and RgpB (FIG. 12A). The Kgp-specific inhibitor Kyt-36 also largely blocks gingipain induced toxicity. The Rgp inhibitor Kyt-1 was also effective but less so (FIG. 8).

### Example 5

Female Balb/c mice were obtained from Harlan Laboratories (USA) and allowed to acclimate. 8 week old mice were challenged orally with 10⁹ CFU W83 P. gingivalis in 2% Na-CMC, 2 times per week for 6 weeks. Control mice received mock challenge with 2% Na-CMC only. 6 weeks after initial infection, mice were sacrificed, perfused and brains dissected. Brains were embedded and sectioned by Neuroscience Associates. 18E6 immunohistochemistry for RgpB showed brain infiltration in 3/6 mice. De Olmos silver stain for neurodegeneration showed staining in 2 of the 3 mice with infiltration (FIG. 7).

### Example 6

DBA-1 mice were obtained from Harlan Laboratories (USA) and allowed to acclimate. Mice were infected using the chamber method (Titanium wire coils (10 mm×5 mm) were implanted subcutaneously in the dorso-lumbar region of each mouse following anesthesia. Incisions were closed using 4.0 G silk sutures and the animals were allowed to rest for 14 days to heal completely to allow for the formation of a fibrous capsule surrounding the coils, thereby, creating chambers into which bacteria P. gingivalis was inoculated) with strain W83 2×(1×10⁸) CFU/ml, at day 0 and again after 4 weeks (into the second chamber, which was implanted after 3 weeks). After 6 weeks, mice were sacrificed, perfused and brains dissected. Brains were embedded and sectioned by Neuroscience Associates. Two of four infected mice showed some level of gingipain infiltration based on immunohistochemistry with 18E6. 1 of 4 mice showed neurodegeneration with silver stain (FIG. 9).

### Example 7

Female Balb/cJ mice were obtained from Taconic and allowed to acclimate. 8 week old mice were challenged orally with 10⁹ CFU W83 P. gingivalis every 3^{rd} day for 4 administrations.

Novel object recognition test for cognitive function was initiated 6 weeks after the initial infection. Mice were familiarized with the test cage for 2 min the day prior to object familiarization. On the day of familiarization, mice were presented with two wooden blue rectangles for 5 minutes. 24 h later mice were presented with one blue rectangle (right side) and one pink heart (left side, both objects made of wood) for the duration of 3 min. The time during which the mouse directed its nose within 2 cm of an object was recorded. Mock infected mice on average spent more time exploring the novel object compared to infected mice who on average spent equal time on both objects indicating cognitive dysfunction (FIG. 8).

### Example 8

Brain sections from aged dogs were provided by Intervivo along with data from cognitive assessments. Dogs were assessed by Intervivo for cognitive impairment as described in Araujo et al, 2011[63]. Brains were embedded and sliced by Neuroscience Associates and assessed for gingipain infiltration as described in Example 1 with 7B9 antibody. All 5 aged dogs with cognitive impairment showed hippocampal staining indicating Lysine gingipain infiltration, while all 3 young dogs showed no staining (FIG. 13). The level of gingpain staining roughly correlates with cognitive impairment. It is expected that treatment of aged dogs with lysine gingipain inhibitors or other treatments targeted to P. gingvalis described herein would block ongoing neurodegeneration and cognitive impairment

### REFERENCES

1. Berisha, F., et al., Retinal abnormalities in early Alzheimer's disease. Invest Ophthalmol Vis Sci, 2007. 48(5): p. 2285-9.
2. Grau, A. J., et al., Periodontal disease as a risk factor for ischemic stroke. Stroke, 2004. 35(2): p. 496-501.
3. Genco, R., S. Offenbacher, and J. Beck, Periodontal disease and cardiovascular disease: epidemiology and possible mechanisms. J Am Dent Assoc, 2002. 133 Suppl: p. 14S-22S.
4. Mealey, B., Diabetes and periodontal diseases. J Periodontol, 1999. 70(8): p. 935-49.
5. Brzozowska, A. and L. Puchalska-Niedbal, [Oral status as a potential source of infection in AMD patients—introduction]. Klin Oczna, 2012. 114(1): p. 29-32.
6. Ercan, E., et al., Evaluation of periodontal pathogens in amniotic fluid and the role of periodontal disease in pre-term birth and low birth weight. Acta Odontol Scand, 2013. 71(3-4): p. 553-9.
7. Koziel, J., P. Mydel, and J. Potempa, The link between periodontal disease and rheumatoid arthritis: an updated review. Curr Rheumatol Rep, 2014. 16(3): p. 408.
8. Gatz, M., et al., Potentially modifiable risk factors for dementia in identical twins. Alzheimers Dement, 2006. 2(2): p. 110-7.
9. Stein, P. S., et al., Tooth loss, dementia and neuropathology in the Nun study. J Am Dent Assoc, 2007. 138(10): p. 1314-22; quiz 1381-2.
10. Noble, J. M., et al., Periodontitis is associated with cognitive impairment among older adults: analysis of NHANES-III. J Neurol Neurosurg Psychiatry, 2009. 80(11): p. 1206-11.
11. Sparks Stein, P., et al., Serum antibodies to periodontal pathogens are a risk factor for Alzheimer's disease. Alzheimers Dement, 2012. 8(3): p. 196-203.
12. Kononen, E., et al., Population-based study of salivary carriage of periodontal pathogens in adults. J Clin Microbiol, 2007. 45(8): p. 2446-51.
13. Davis, P. R. and E. Head, Prevention approaches in a preclinical canine model of Alzheimer's disease: benefits and challenges. Front Pharmacol, 2014. 5: p. 47.
14. Berglundh, T., J. Lindhe, and J. D. Sterrett, Clinical and structural characteristics of periodontal tissues in young and old dogs. J Clin Periodontol, 1991. 18(8): p. 616-23.
15. Albuquerque, C., et al., Canine periodontitis: the dog as an important model for periodontal studies. Vet J, 2012. 191(3): p. 299-305.
16. Kataoka, S., et al., A novel, potent dual inhibitor of Arg-gingipains and Lys-gingipain as a promising agent for periodontal disease therapy. FASEB J, 2014.
17. Nguyen, K. A., J. Travis, and J. Potempa, Does the importance of the C-terminal residues in the maturation of RgpB from Porphyromonas gingivalis reveal a novel mechanism for protein export in a subgroup of Gram-Negative bacteria? J Bacteriol, 2007. 189(3): p. 833-43.
18. Sztukowska, M., et al., The C-terminal domains of the gingipain K polyprotein are necessary for assembly of the active enzyme and expression of associated activities. Mol Microbiol, 2004. 54(5): p. 1393-408.
19. Booth, V., F. P. Ashley, and T. Lehner, Passive immunization with monoclonal antibodies against Porphyromonas gingivalis in patients with periodontitis. Infect Immun, 1996. 64(2): p. 422-7.
20. Poole, S., et al., Determining the presence of periodontopathic virulence factors in short- term postmortem Alzheimer's disease brain tissue. J Alzheimers Dis, 2013. 36(4): p. 665-77.
21. Ishida, N., et al., Periodontitis induced by bacterial infection exacerbates features of Alzheimer's disease in transgenic mice. Alzheimer's & Dementia: The Journal of the Alzheimer's Association. 9(4): p. P851.
22. Poole, S., et al., Active Invasion of Porphyromonas gingivalis and Infection-Induced Complement Activation in ApoE−/−Mice Brains. J Alzheimers Dis, 2014.
23. Guo, Y., K. A. Nguyen, and J. Potempa, Dichotomy of gingipains action as virulence factors: from cleaving substrates with the precision of a surgeon's knife to a meat chopper-like brutal degradation of proteins. Periodontol 2000, 2010. 54(1): p. 15-44.
24. Travis, J., A. Banbula, and J. Potempa, The role of bacterial and host proteinases in periodontal disease. Adv Exp Med Biol, 2000. 477: p. 455-65.
25. Byrne, S. J., et al., Progression of chronic periodontitis can be predicted by the levels of Porphyromonas gingivalis and Treponema denticola in subgingival plaque. Oral Microbiol Immunol, 2009. 24(6): p. 469-77.
26. Mahendra, J., et al., Prevalence of periodontal pathogens in coronary atherosclerotic plaque of patients undergoing coronary artery bypass graft surgery. J Maxillofac Oral Surg, 2009. 8(2): p. 108-13.
27. Stelzel, M., et al., Detection of Porphyromonas gingivalis DNA in aortic tissue by PCR. J Periodontol, 2002. 73(8): p. 868-70.
28. Ishikawa, M., et al., Oral Porphyromonas gingivalis translocates to the liver and regulates hepatic glycogen synthesis through the Akt/GSK-3beta signaling pathway. Biochim Biophys Acta, 2013. 1832(12): p. 2035-2043.
29. Guo, L., J. Duggan, and M. F. Cordeiro, Alzheimer's disease and retinal neurodegeneration. Curr Alzheimer Res, 2010. 7(1): p. 3-14.
30. Kaarniranta, K., et al., Age-related macular degeneration (AMD): Alzheimer's disease in the eye? J Alzheimers Dis, 2011. 24(4): p. 615-31.
31. Toda, R., et al., Comparison of drug permeabilities across the blood-retinal barrier, blood-aqueous humor barrier, and blood-brain barrier. J Pharm Sci, 2011. 100(9): p. 3904-11.
32. Sivak, J. M., The aging eye: common degenerative mechanisms between the Alzheimer's brain and retinal disease. Invest Ophthalmol Vis Sci, 2013. 54(1): p. 871-80.
33. Harvey, H. and S. Durant, The role of glial cells and the complement system in retinal diseases and Alzheimer's disease: common neural degeneration mechanisms. Exp Brain Res, 2014.
34. Stathopoulou, P. G., et al., Porphyromonas gingivalis induce apoptosis in human gingival epithelial cells through a gingipain-dependent mechanism. BMC Microbiol, 2009. 9: p. 107.
35. Sheets, S. M., et al., Gingipains from Porphyromonas gingivalis W83 induce cell adhesion molecule cleavage and apoptosis in endothelial cells. Infect Immun, 2005. 73(3): p. 1543-52.
36. Sheets, S. M., et al., Gingipains from Porphyromonas gingivalis W83 synergistically disrupt endothelial cell adhesion and can induce caspase-independent apoptosis. Infect Immun, 2006. 74(10): p. 5667-78.
37. Aduse-Opoku, J., et al., Generation of lys-gingipain protease activity in Porphyromonas gingivalis W50 is independent of Arg-gingipain protease activities. Microbiology, 2000. 146 (Pt 8): p. 1933-40.
38. Graves, D. T., et al., The use of rodent models to investigate host-bacteria interactions related to periodontal diseases. J Clin Periodontol, 2008. 35(2): p. 89-105.
39. Kadowaki, T., et al., Suppression of pathogenicity of Porphyromonas gingivalis by newly developed gingipain inhibitors. Mol Pharmacol, 2004. 66(6): p. 1599-606.
40. Walmsley, S., et al., Lopinavir-ritonavir versus nelfinavir for the initial treatment of HIV infection. N Engl J Med, 2002. 346(26): p. 2039-46.
41. Marzolini, C., et al., The brain entry of HIV-1 protease inhibitors is facilitated when used in combination. Mol Pharm, 2013. 10(6): p. 2340-9.
42. Abedon, S. T., et al., Phage treatment of human infections. Bacteriophage, 2011. 1(2): p. 66-85.
43. Dubos, R. J., J. H. Straus, and C. Pierce, THE MULTIPLICATION OF BACTERIOPHAGE IN VIVO AND ITS PROTECTIVE EFFECT AGAINST AN EXPERIMENTAL INFECTION WITH SHIGELLA DYSENTERIAE. J Exp Med, 1943. 78(3): p. 161-8.
44. Machuca, P., et al., Isolation of a novel bacteriophage specific for the periodontal pathogen Fusobacterium nucleatum. Appl Environ Microbiol, 2010. 76(21): p. 7243-50.
45. Hunt, D. E., J. V. Jones, and V. R. Dowell, Selective medium for the isolation of Bacteroides gingivalis. Journal of Clinical Microbiology, 1986. 23(3): p. 441-445.
46. Gill, J. J. and P. Hyman, Phage choice, isolation, and preparation for phage therapy. Curr Pharm Biotechnol, 2010. 11(1): p. 2-14.
47. Genco, C. A., et al., A peptide domain on gingipain R which confers immunity against Porphyromonas gingivalis infection in mice. Infect Immun, 1998. 66(9): p. 4108-14.
48. Gibson, F. C., 3rd and C. A. Genco, Prevention of Porphyromonas gingivalis-induced oral bone loss following immunization with gingipain R1. Infect Immun, 2001. 69(12): p. 7959-63.
49. Page, R. C., et al., Immunization of Macaca fascicularis against experimental periodontitis using a vaccine containing cysteine proteases purified from Porphyromonas gingivalis. Oral Microbiol Immunol, 2007. 22(3): p. 162-8.
50. Guo, M., et al., kgp, rgpA, and rgpB DNA vaccines induce antibody responses in experimental peri-implantitis. J Periodontol, 2014. 85(11): p. 1575-81.
51. Zhu, C., et al., Induction of immune response and prevention of alveolar bone loss with recombinant Porphyromonas gingivalis peptidylarginine deiminase. Arch Oral Biol, 2013. 58(12): p. 1777-83.
52. Kesavalu, L., et al., Porphyromonas gingivalis virulence in mice: induction of immunity to bacterial components. Infect Immun, 1992. 60(4): p. 1455-64.
53. Wang, L., et al., Subcutaneous vaccination with Porphyromonas gingivalis ameliorates periodontitis by modulating Th17/Treg imbalance in a murine model. Int Immunopharmacol, 2015.
54. Genco, C. A., et al., Influence of immunization on Porphyromonas gingivalis colonization and invasion in the mouse chamber model. Infection and Immunity, 1992. 60(4): p. 1447-1454.
55. McClellan, D. L., A. L. Griffen, and E. J. Leys, Age and prevalence of Porphyromonas gingivalis in children. J Clin Microbiol, 1996. 34(8): p. 2017-9.
56. Petit, M. D., et al., Epidemiology and transmission of Porphyromonas gingivalis and
Actinobacillus actinomycetemcomitans among children and their family members. A report of 4 surveys. J Clin Periodontol, 1993. 20(9): p. 641-50.
57. Petit, M. D., et al., Prevalence of periodontitis and suspected periodontal pathogens in families of adult periodontitis patients. J Clin Periodontol, 1994. 21(2): p. 76-85.
58. Tuite-McDonnell, M., et al., Concordance of Porphyromonas gingivalis colonization in families. J Clin Microbiol, 1997. 35(2): p. 455-61.
59. Faux, N. G., et al., An anemia of Alzheimer's disease. Mol Psychiatry, 2014.
60. Dashper, S. G., et al., Hemoglobin hydrolysis and heme acquisition by Porphyromonas gingivalis. Oral Microbiol Immunol, 2004. 19(1): p. 50-6.
61. Tian, H., et al., Radio-deoxynucleoside Analogs used for Imaging tk Expression in a Transgenic Mouse Model of Induced Hepatocellular Carcinoma. Theranostics, 2012. 2(6): p. 597-606.
62. •, S.S.Y.P.D.M.C. and C. Goldsbury, Human Chorionic Gonadotropin Increases b- Cleavage of Amyloid Precursor Protein in SH-SY5Y Cells. Cell Mol Neurobiol 2013. 33(747-751).
63. Araujo, J. A., et al., Cholinesterase inhibitors improve both memory and complex learning in aged beagle dogs. J Alzheimers Dis, 2011. 26(1): p. 143-55.

### CONCLUSION

Those of skill in the art should, in light of the present disclosure, appreciate that many changes can be made in the specific embodiments which are disclosed and still obtain a like or similar result without departing from the spirit and scope of the invention. The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein, will become apparent to those skilled in the art from the foregoing description. Accordingly, the invention is limited only by the following claims. All of the foregoing references are hereby incorporated by reference herein.

1. A method of treating a brain disorder comprising administering to a subject in need thereof a therapeutically effective amount of a pharmaceutically acceptable agent which inhibits Lysine Gingipain (Kgp) activity.

2. The method of claim 1, wherein the brain disorder is Alzheimer's disease.

3. (canceled)

4. The method of claim 1, wherein the pharmaceutically acceptable agent is at least 30 times more selective for Kgp than for trypsin and cathepsin L.

5-10. (canceled)

11. The method of claim 1, wherein the pharmaceutically acceptable agent which inhibits Kgp activity is delivered orally, intranasally, subcutaneously, intravenously, intracranially, or topically to the eye.

12-13. (canceled)

14. The method of claim 1, wherein the subject is an animal or a human.

15-20. (canceled)

21. The method of claim 1, further comprising administration of one or more additional therapeutic agent(s) indicated for the treatment and/or prevention of brain disorders (e.g., Alzheimer's disease).

22. The method of claim 1, wherein the agent which inhibits Kgp activity is a compound of formula (I): 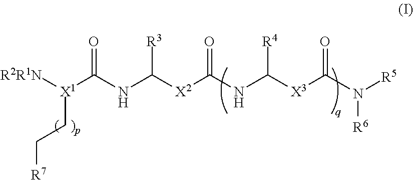
wherein
R¹, R², R⁵, and R⁶ is each independently a bond, hydrogen, an amino protecting group, hydroxyl, COOH, COH, carbonylaminoethylanilinyl, an alkyl, a cycloalkyl, an alkenyl, an aryl, an alkylaryl, an arylalkyl, an alkoxyalkyl, an alkoxyaryl, an alkoxyalkylaryl, an alkoxyarylalkyl, an alkyloxycarbonyl, a carboxyalkyl, a carboxyaryl, a carboxyalkylaryl, a carboxyarylalkyl, a heterocycle radical, an oxycarbonyl, benzyloxycarbonyl, amido, methylphenyamide, methylphenylamine, amine, carboxyl, alkyloxycarbonyl, or a side chain of an α-amino acid;
where in R¹, R², R⁵, and R⁶, each of the carbonylaminoethylanilinyl, the alkyl; the cycloalkyl, the alkenyl, the aryl, the alkylaryl, the arylalkyl, the alkoxyalkyl, the alkoxyaryl, the alkoxyalkylaryl, the alkoxyarylalkyl, the alkyloxycarbonyl, the carboxyalkyl, the carboxyaryl, the carboxyalkylaryl, the carboxyarylalkyl, the heterocycle radical, the oxycarbonyl, the benzyloxycarbonyl, the amido, the methylphenyamide, the methylphenylamine, the amine, the carboxyl, the alkyloxycarbonyl, and the side chain of the α-amino acid is independently unsubstituted or substituted with one or more of amino, amide, halogen, hydroxyl, amitidine, a lower alkoxy, a lower carboxy, a lower alkyl, aryl, —NR⁸R⁹, a loweralkoxycarbonylamino, or a protecting group;
R³ and R⁴ is each independently a bond, hydrogen, hydroxyl, —COOH, —COH, —(CH₂)ₙ—NR⁸R⁹, an alkyl, an aminoalkyl, a cycloalkyl, an aryl, an alkylaryl, an arylalkyl, an alkoxyalkyl, an alkoxyaryl, an alkoxyalkylaryl, an alkoxyarylalkyl, a alkyloxycarbonyl, a carboxyalkyl, a carboxyaryl, a carboxyalkylaryl, a carboxyarylalkyl, a heterocycle radical, oxycarbonyl, amido, carboxyl, guanidine, a side chain of an α-amino acid, isobutyl, carbamoylmethyl, 2-carboxyethyl, 4-aminobutyl, or benzyl;
where in R³ and R⁴, each of the hydroxyl, the —(CH₂)ₙ—NR⁸R⁹, the alkyl, the aminoalkyl, the cycloalkyl, the aryl, the alkylaryl, the arylalkyl, the alkoxyalkyl, the alkoxyaryl, the alkoxyalkylaryl, the alkoxyarylalkyl, the alkyloxycarbonyl, the carboxyalkyl, the carboxyaryl, the carboxyalkylaryl, the carboxyarylalkyl, the heterocycle radical, the oxycarbonyl, the amido, the carboxyl, the guanidine, the side chain of the α-amino acid, the isobutyl, the carbamoylmethyl, the 2-carboxyethyl, the 4-aminobutyl, and benzyl is independently unsubstituted or substituted with one or more of amino, amide, halogen, hydroxyl, amitidine, a lower alkoxy, a lower carboxy, a lower alkyl, aryl, a loweralkoxycarbonylamino, or a protecting group;
R⁷ is a bond, hydrogen, hydroxyl, COOH, COR⁸, an alkyl, a cycloalkyl, an aryl, an alkylaryl, an arylalkyl, an alkoxyalkyl, an alkoxyaryl, an alkoxyalkylaryl, an alkoxyarylalkyl, an alkyloxycarbonyl, a carboxyalkyl, a carboxyaryl, a carboxyalkylaryl, a carboxyarylalkyl, a heterocycle radical, oxycarbonyl, amino, methylamino, amido, dimethylamino, (2-aminoethyl)amino, 1,1-dimethylhydrazino, 1-methyl-1-phenylhydrazino, or benzyloxycarbonyl, a side chain of an α-amino acid, or carboxyl;
where in R⁷, each of the COR⁸, the alkyl, the cycloalkyl, the aryl, the alkylaryl, the arylalkyl, the alkoxyalkyl, the alkoxyaryl, the alkoxyalkylaryl, the alkoxyarylalkyl, alkyloxycarbonyl, the carboxyalkyl, the carboxyaryl, the carboxyalkylaryl, the carboxyarylalkyl, the heterocycle radical, the oxycarbonyl, the amino, the methylamino, the amido, the dimethylamino, the (2-aminoethyl)amino, the 1,1-dimethylhydrazino, the 1-methyl-1-phenylhydrazino, the benzyloxycarbonyl, the carboxyl, and the side chain of the α-amino acid is independently unsubstituted or substituted with one or more of amino, amide, halogen, amitidine, hydroxyl, a lower alkoxy, a lower carboxy, a lower alkyl (C₁-C₇), —NR⁹R¹⁰, aryl, loweralkoxycarbonylamino, or a protecting group;
R⁸, R⁹ and R¹⁰ is each independently a bond, hydrogen, hydroxyl, —COOH, —COH, —NH, —NH₂, —CNHNH₂, —CNH₂NNO₂, an alkyl, a cycloalkyl, an aryl, an alkylaryl, an arylalkyl, an alkoxyalkyl, an alkoxyaryl, an alkoxyalkylaryl, an alkoxyarylalkyl, a carboxyalkyl, a carboxyaryl, a carboxyalkylaryl, a carboxyarylalkyl, a heterocycle radical, oxycarbonyl, an alkyloxycarbonyl, an amino, hydrazine, or a side chain of an α-amino acid;
where in R⁸, R⁹, and R¹⁰, each of the alkyl, the cycloalkyl, the aryl, the alkylaryl, the arylalkyl, the alkoxyalkyl, the alkoxyaryl, the alkoxyalkylaryl, the alkoxyarylalkyl, the carboxyalkyl, the carboxyaryl, the carboxyalkylaryl, the carboxyarylalkyl, the heterocycle radical, the oxycarbonyl, the alkyloxycarbonyl, the hydrazine, the amino, the side chain of the α-amino acid independently unsubstituted or substituted with one or more of amino, amide, hydroxyl, halogen, amitidine, a lower alkoxy, a lower carboxy, a lower alkyl, aryl, loweralkoxycarbonylamino, or a protecting group;
X¹ is —CH—;
X² and X³ are each independently a bond, —CH—, —O—, —S—, N, —CHOH—, —COO— or —CO—;
n is an integer from 1 to 6;
p is an integer from 0 to 4; and
q is an integer from 0 to 2,
or a pharmaceutically acceptable salt thereof.

23. The method of claim 22, wherein
R¹ or R² is unsubstituted or substituted benzyloxycarbonyl;
R³ and R⁴ is each independently a bond, hydrogen, unsubstituted or substituted hydroxyl, unsubstituted or substituted carboxyl, an unsubstituted or substituted aminoalkyl, or an unsubstituted or substituted side chain of an α-amino acid;
R⁵ and R⁶ is independently a bond, H, unsubstituted or substituted hydroxyl, unsubstituted or substituted carboxyl, unsubstituted or substituted lower alkyl, or unsubstituted or substituted alkylaryl;
R⁷ is unsubstituted or substituted alkyl amine, unsubstituted or substituted 1-methyl-1-phenyl-hydrozinocarbonyl, or unsubstituted or substituted alkylcarbonyl;
X₂ and X₃ are both —CO—;
p is an integer from 1 to 4; and
q is 1.

24. The method of claim 1, wherein the agent which inhibits Kgp activity is a compound of formula (II): 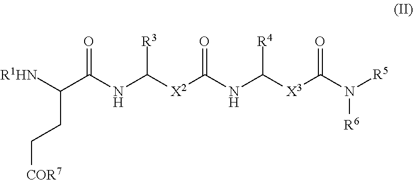
or a pharmaceutically acceptable salt thereof, wherein:
X² and X³ is each independently a bond, —CHOH— or —CO—;
R¹ and R⁷ is each independently hydrogen, an optionally substituted carboxyl group, hydroxyl or substituted oxycarbonyl;
R³ is substituted oxycarbonyl, optionally substituted alkyl or optionally substituted aminoalkyl;
R⁴ is optionally substituted alkyl, optionally substituted aminoalkyl, hydroxyl, lower alkoxy, optionally substituted piperazinyl or the like;
R⁵ is optionally substituted alkyl, hydrogen or a R-group side chain of an .alpha.-amino acid optionally protected by a protective group; and
R⁶ is optionally substituted alkyl, hydroxyl, or lower alkoxy.

25-29. (canceled)

30. The method of claim 22, wherein the agent which inhibits Kgp activity is Kyt-36 having the structure:

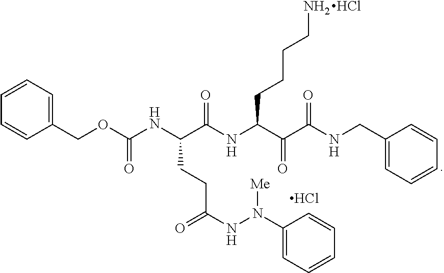 [SMILES: Cl ; CN(NC(=O)CC[C@H](NC(=O)OCc1ccccc1)C(=O)N[C@@H](CCCCN)C(=O)C(=O)NCc1ccccc1)c1ccccc1 ; Cl]

31. The method of claim 24, wherein the agent which inhibits Kgp activity is Kyt-41 having the structure:

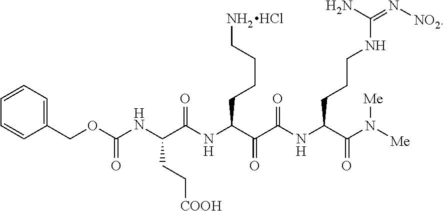 [SMILES: CN(C)C(=O)[C@H](CCCN/C(N)=N\[N+](=O)[O-])NC(=O)C(=O)[C@H](CCCCN)NC(=O)[C@H](CCC(=O)O)NC(=O)OCc1ccccc1 ; Cl]

32-34. (canceled)

35. The method of claim 1, wherein the disorder is selected from the group consisting of Alzheimer's disease, Down's syndrome, epilepsy, autism, Parkinson's disease, essential tremor, fronto-temporal dementia, progressive supranuclear palsy, amyotrophic lateral sclerosis, Huntington's disease, multiple sclerosis, mild cognitive impairment, age associated memory impairment, chronic traumatic encephalopathy, stroke, Lewy Body disease, multiple system atrophy, schizophrenia and depression.

36-41. (canceled)

42. The method of claim 1, wherein the agent which inhibits Kgp activity is an antibody targeting gingipains or an antibody fragment targeting gingipains.

43. The method of claim 42, wherein the agent which inhibits Kgp activity is selected from 7B9 and 15C8.

44-95. (canceled)

96. The method of claim 1, wherein the pharmaceutically acceptable agent has a Ki of Lysine Gingipain (Kgp) of less than or equal to 10 nanomolar (nM).
